SEQUENCE LISTING (23 sequences)

SEQ ID NO: 1 - protein, 159 aa, Escherichia coli
MISLIAALAVDHVIGMENAMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 2 - protein, 245 aa, Homo sapiens
SLALSLTADQMVSALLDAEPPILYSEYDPTRPFSEASMMGLLTNLADRELVHMINWAKRVPGFVDLALHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSSKRMEHLYSMKCKNVVPLSDLLLEMLDAHRL

SEQ ID NO: 3 - protein, 108 aa, Homo sapiens
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 4 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 5 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - DNA, 660 bp, Homo sapiens
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 7 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - DNA, 987 bp, Homo sapiens
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 9 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 10 - DNA, 435 bp, Homo sapiens
atgtggctgcagagcctgctgctcttgggcactgtggcctgcagcatctctgcacccgcccgctcgcccagccccagcacacagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaaacagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 11 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 12 - DNA, 1434 bp, Listeria monocytogenes
atgaatatgaaaaaagcaactatcgcggctacagctgggattgcggtaacagcatttgctgctccaacaatcgcatccgcaagcactgtagtagttgaagctggtgatactctttggggtatcgcacaaagtaaagggactactgttgacgcaattaaaaaagcaaacaatttaacaacagataaaatcgtaccaggtcaaaaattacaagtaaatgaggttgctgctgctgaaaaaacagagaaatctgttagcgcaacttggttaaacgttcgtagtggcgctggtgttgataacagtattattacgtcaatcaaaggcggaacaaaagtaactgttgaatcaaccgaatctaatggctggaacaaaattacttacaacgacggggaaactggtttcgttaacggtaaatacttaactgacaaagtagcaagcactcctgttgcaccaacacaagaagtgaaaaaagaaactactattcaacaagctgcacctgctgcagaaacaaaaactgaagtaaaacaaactacacaagcaactacacctgcacctaaagtagcagaaacgaaagaaactccagtggtagatcaaaatgctactacacacgctgttaaaagcggtgacactatttgggctttatccgtaaaatacggtgtttccgttcaagacattatgtcatggaataatttatcttcttcttctatttatgtaggtcaaaaacttgctattaaacaaacagccaacacagttactccaaaagcagaagtgaaaaaagaagctccagcagctgaaaaacaagctgcaccagtagttaaagaaaatactaacactaacacaaatactactacagagaaaaaagaaacaacacaacaacaaacagcacctaaagcaccaacagaagctgcaaaaccagctcccgcaccatctacaaacacaaatgctaataaaacgaatacgaatacaaacaatactaatacaagtacaccatctaaaaataccaatactaatacaaactccaatacgaatacaaactcaaatacgaatgctaatcaaggttcttctaacaataacagcaattcaagtgcaagtgctattattgctgaagctcaaaaacaccttggaaaagcttattcatggggtggtaacggaccaactacatttgattgctctggttacactaaatatgtatttgctaaagcgggaatctcccttccacgtacttctggcgcacaatacgctagcactacaagaatctctgaatctcaagcaaaacctggtgatttagtattctttgactatggtagcggaatttctcacgttggtatctacgttggtaatggtcaaatgattaacgcgcaagacaatggcgttaaatacgataacatccacggctctggctggggtaaatatctagttggcttcggtcgcgtataa

SEQ ID NO: 13 - protein, 477 aa, Listeria monocytogenes
MNMKKATIAATAGIAVTAFAAPTIASASTVVVEAGDTLWGIAQSKGTTVDAIKKANNLTTDKIVPGQKLQVNEVAAAEKTEKSVSATWLNVRSGAGVDNSIITSIKGGTKVTVESTESNGWNKITYNDGETGFVNGKYLTDKVASTPVAPTQEVKKETTIQQAAPAAETKTEVKQTTQATTPAPKVAETKETPVVDQNATTHAVKSGDTIWALSVKYGVSVQDIMSWNNLSSSSIYVGQKLAIKQTANTVTPKAEVKKEAPAAEKQAAPVVKENTNTNTNTTTEKKETTQQQTAPKAPTEAAKPAPAPSTNTNANKTNTNTNNTNTSTPSKNTNTNTNSNTNTNSNTNANQGSSNNNSNSSASAIIAEAQKHLGKAYSWGGNGPTTFDCSGYTKYVFAKAGISLPRTSGAQYASTTRISESQAKPGDLVFFDYGSGISHVGIYVGNGQMINAQDNGVKYDNIHGSGWGKYLVGFGRV

SEQ ID NO: 14 - protein, 477 aa, Listeria monocytogenes; identical to SEQ ID NO: 13

SEQ ID NO: 15 - DNA, 1488 bp, Salmonella enterica
atggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatatcgatctgaagcagatcaactctcagaccctgggtctggatacgctgaatgtgcaacaaaaatataaggtcagcgatacggctgcaactgttacaggatatgccgatactacgattgctttagacaatagtacttttaaagcctcggctactggtcttggtggtactgaccagaaaattgatggcgatttaaaatttgatgatacgactggaaaatattacgccaaagttaccgttacggggggaactggtaaagatggctattatgaagtttccgttgataagacgaacggtgaggtgactcttgctggcggtgcgacttccccgcttacaggtggactacctgcgacagcaactgaggatgtgaaaaatgtacaagttgcaaatgctgatttgacagaggctaaagccgcattgacagcagcaggtgttaccggcacagcatctgttgttaagatgtcttatactgataataacggtaaaactattgatggtggtttagcagttaaggtaggcgatgattactattctgcaactcaaaataaagatggttccataagtattaatactacgaaatacactgcagatgacggtacatccaaaactgcactaaacaaactgggtggcgcagacggcaaaaccgaagttgtttctattggtggtaaaacttacgctgcaagtaaagccgaaggtcacaactttaaagcacagcctgatctggcggaagcggctgctacaaccaccgaaaacccgctgcagaaaattgatgctgctttggcacaggttgacacgttacgttctgacctgggtgcggtacagaaccgtttcaactccgctattaccaacctgggcaacaccgtaaacaacctgacttctgcccgtagccgtatcgaagattccgactacgcgaccgaagtttccaacatgtctcgcgcgcagattctgcagcaggccggtacctccgttctggcgcaggcgaaccaggttccgcaaaacgtcctctctttactgcgttaa

SEQ ID NO: 16 - protein, 495 aa, Salmonella enterica [sequence omitted]

SEQ ID NO: 17 - DNA, 3000 bp, Unknown
  flagellin-grp170
atggcagctacagtaaggaggcagaggccaaggaggctactctgttgggccttggtggctgtcctcttggcagacctgttggcactgagcgacacattggctgtgatgtctgtagacctgggcagtgaatccatgaaggtggccattgtcaagcctggagtgcccatggagattgtattgaacaaggaatctcggaggaaaactccagtgactgtgaccttgaaagaaaatgaaaggtttttaggtgatagtgcagccggcatggccatcaagaacccaaaggctacgctccgttatttccagcacctccttggaaaacaggcggataaccctcatgtggccctttaccggtcccgtttcccagaacatgagctaattgttgacccacagaggcagactgtgcgcttccagatcagtccgcagctgcagttctctcccgaggaggtactgggcatggttctgaactactcccgttccttggctgaagattttgctgaacaacccattaaggatgcagtgatcaccgtgccagcctttttcaaccaggctgagcgccgagctgtgctgcaggctgctcggatggctggcctcaaggtgctgcagctcatcaatgacaacactgccacagccctcagctacggtgtcttccgccggaaagatatcaattccactgcacagaacgtcatgttctatgacatgggctcgggcagcactgtgtgcaccatcgtcacctaccagacagtgaagactaaggaggctgggatgcaaccacagctgcagatccggggcgtgggatttgaccgcaccctgggtggcctggagatggagcttcggcttcgagaacacctggctaagctcttcaatgagcagcgcaagggccagaaagccaaggatgttcgggaaaacccccgggccatggccaaactgcttcgggaagccaaccggcttaaaaccgtcctgagtgccaacgctgatcacatggcacagattgagggcttgatggatgatgtggacttcaaggccaaagtaactcgagtggaattcgaggagctgtgtgcagatttgtttgaccgtgtgcctggacctgtgcagcaggccttgcagagtgcagagatgagcttggatcaaattgagcaggtgatcctggtgggcggggccactcgtgttcccaaagttcaagaagtgctgctcaaggccgtgggcaaggaggaactaggaaagaacatcaatgcggacgaagctgctgccatgggggctgtgtaccaggcagcggcgctcagcaaggccttcaaagtgaagccatttgttgtgcgggatgctgtcatttacccaatcctggtggagttcacaagggaggtggaggaggagcctgggctccgaagcctgaaacacaataagcgtgtgctcttctcccgaatggggccctaccctcagcgcaaagtcatcacctttaaccgctacagccatgatttcaacttccacatcaactacggtgacctgggcttcctggggcctgaggatcttcgggtatttggctcccagaatctgaccacagtaaaactaaaaggcgtgggagagagcttcaagaaatatcccgactatgagtccaaaggcatcaaggcccactttaacctggatgagagtggcgtgctcagtttagacagggtggagtccgtatttgagaccctggtggaggatagcccagaggaagaatctactcttaccaaacttggcaacaccatatccagcctgtttggaggtggtacctcatcagatgccaaagagaatggtactgatgctgtacaggaggaagaggagagccccgctgaggggagcaaggatgagcctgcagagcagggggaactcaaggaagaagctgaacccccagcagaggagacctctcagcctccaccctctgagcctaagggggatgcagcccgtgagggagagaaacctgatgaaaaagagagtggggacaagcctgaggcccagaagcccaatgagaaggggcaagcagggcctgagggtgctgctccagctcctgaggaggacaaaaagccgaaacctgcccggaagcagaaaatggtggaggagataggtgtggagctggctgtcttggacctgcctgacttgccagaggatgagctggcccgttctgtgcagaaacttgaagaactgaccctgcgcgacctagagaagcaggagagggagaaagctgccaacagcttggaggctttcatctttgagacccaggacaagctgtaccagcctgagtaccaggaagtgtccactgaggaacagcgggaggagatctcggggaaactcagcgccacttctacctggctggaggatgagggatttggagccaccactgtgatgctgaaggacaagctggctgagctgagaaagctgtgccaagggctgttttttcgggtggaagaacgcaggaaatggccagagcggctttcagctctggataatctcctcaaccattccagcattttcctcaagggtgcccggctcatcccggagatggaccaggtcttcactgaagtggagatgacgacattagagaaagttatcaatgacacctgggcctggaagaatgcaactctggccgaacaagccaagcttcctgccacagagaagcctgtgctgctttcaaaagacattgaggccaaaatgatggccctggaccgggaggtacagtatctactcaataaggccaagtttaccaagccacggccacggcccaaagacaagaatggcacccgggcagaacctcccctcaatgccagtgctggtgaccaagaggagaaggtcattccacctgcaggccagactgaagaggcgaaacccattttagaacctgacaaagaagagactggtacggaaccagcagactcggagcctctggaattaggaggtcctggagctggacctgaacaggaagagcagtcagcaggacagaagcggccttcaaagaacgatgaactataa

SEQ ID NO: 18 - protein, 999 aa, Unknown
  flagellin-grp170
MADKVRRQRPRRRVCWALVAVLLADLLALSDTLAVMSVDLGSESMKVAIVKPGVPMEIVLNKESRRKTPVIVTLKENERFFGDSAASMAIKNPKATLRYFQHLLGKQADNPHVALYQARFPEHELTFDPQRQTVHFQISSQLQFSPEEVLGMVLNYSRSLAEDFAEQPIKDAVITVPVFFNQAERRAVLQAARMAGLKVLQLINDNTATALSYGVFRRKDINTTAQNIMFYDMGSGSTVCTIVTYQMVKTKEAGMQPQLQIRGVGFDRTLGGLEMELRLRERLAGLFNEQRKGQRAKDVRENPRAMAKLLREANRLKTVLSANADHMAQIEGLMDDVDFKAKVTRVEFEELCADLFERVPGPVQQALQSAEMSLDEIEQVILVGGATRVPRVQEVLLKAVGKEELGKNINADEAAAMGAVYQAAALSKAFKVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPLKNDEL

SEQ ID NO: 19 - protein, 830 aa, Unknown
  flagellin-grp170
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTATTTENPLQKIDAALAQVDTLRSDLGAVQNRFNSAITNLGNTVNNLTSARSRIEDSDYATEVSNMSRAQILQQAGTSVLAQANQVPQNVLSLLRKVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPL

SEQ ID NO: 20 - DNA, 3481 bp, Artificial Sequence
  Synthetic
tattaaaatcttaataggttttgataaaagcgaacgtggggaaacccgaaccaaaccttcttctaaattctctctcatctctcttaaagcaaacttctctcttgtctttcttgcatgagcgatcttcaacgttgtcagatcgtgcttcggcaccagtacaatgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgaaatggaaagcattatgagccgtggtattccttcaggaattttggaggaaaaagctattcagttcaaacgtgccaaagaagggaataaacccttgaaggatgagattcccaagcctgaggatatgtatgtgtctcacacttctaaatggaatgtgctcagaaaaatgagccaaaagactgtggatctttccaaagcagctgctgggatgggattcatcaataagcatatgcttacgggcaacatcttggcacaaccaacaacagtcttggatattcccgtcacaaaggataaaacacttgcgatggccagtgattttattcgtaaggagaatctcaagacttctgccattcacattggagcaattgagattattatccagagctttgcttcccctgaaagtgatttgatgggaggctttttgcttgtggattctttacacactgatacagctaatgctattcgtagcatttttgttgctccaatgcggggaggaagaccagtcagagtggtgaccttcccaaatacactggcacctgtatcatgtgatctgaacaatagattcaagctcatttgctcattgccaaactgtgatattgtccagggtagccaagtagcagaagtgagtgtaaatgttgcaggatgtgctacttccatagagaaatctcacaccccttcccaattgtatacagaggaatttgaaaaggagggtgctgttgttgtagaatacttaggcagacagacctattgtgctcagcctagcaatttacccacagaagaaaaacttcggtcccttaagtttgactttcatgttgaacaaccaagtgtcctgaagttatccaattcctgcaatgcgcactttgtcaagggagaaagtttgaaatactctatttctggcaaagaagcagaaaaccatgcagttcatgctactgtggtctctcgagaaggggcttctgcggcacccaagcaatatgatcctattttgggacgggtgctggatccacgaaatgggaatgtggcttttccacaaatggagcaaaacttgtttgccctttctttggatgatacaagctcagttcgtggttctttgcttgacacaaaattcgcacaaactcgagttttgttgtccaaggctatggctggtggtgatgtgttattggatgagtatctctatgatgtggtcaatggacaagattttagagctactgtcgcttttttgcgcacccatgttataacaggcaaaataaaggtgacagctaccaccaacatttctgacaactcgggttgttgtttgatgttggccataaatagtggtgtgaggggtaagtatagtactgatgtttatactatctgctctcaagactccatgacgtggaacccagggtgcaaaaagaacttctcgttcacatttaatccaaacccttgtggggattcttggtctgctgagatgataagtcgaagcagagttaggatgacagttatttgtgtttcgggatggaccttatctcctaccacagatgtgattgccaagctagactggtcaattgtcaatgagaaatgtgagcccaccatttaccacttggctgattgtcagaattggttaccccttaatcgttggatgggaaaattgacttttccccagggtgtgacaagtgaggttcgaaggatgcctctttctataggaggcggtgctggtgcgactcaagctttcttggccaatatgcccaattcatggatatcaatgtggagatattttagaggtgaacttcactttgaagttactaaaatgagctctccatatattaaagccactgttacatttctcatagcttttggtaatcttagtgatgcctttggtttttatgagagttttcctcatagaattgttcaatttgctgaggttgaggaaaaatgtactttggttttctcccaacaagagtttgtcactgcttggtcaacacaagtaaaccccagaaccacacttgaagcagatggttgtccctacctatatgcaattattcatgatagtacaacaggtacaatctccggagattttaatcttggggtcaagcttgttggcattaaggatttttgtggtataggttctaatccgggtattgatggttcccgcttgcttggagctatagcacaaggacctgtttgtgctgaagcctcagatgtgtatagcccatgtatgatagctagcactcctcctgctccattttcagacgttacagcagtaacttttgacttaatcaacggcaaaataactcctgttggtgatgacaattggaatacgcacatttataatcctccaattatgaatgtcttgcgtactgctgcttggaaatctggaactattcatgttcaacttaatgttaggggtgctggtgtcaaaagagcagattgggatggtcaagtctttgtttacctgcgccagtccatgaaccctgaaagttatgatgcgcggacatttgtgatctcacaacctggttctgccatgttgaacttctcttttgatatcatagggccgaatagcggatttgaatttgccgaaagcccatgggccaatcagaccacctggtatcttgaatgtgttgctaccaatcccagacaaatacagcaatttgaggtcaacatgcgcttcgatcctaatttcagggttgccggcaatatcctgatgcccccatttccactgtcaacggaaactccaccgttattaaagtttaggtttcgggatattgaacgctccaagcgtagtgttatggttggacacactgctactgctgcttaactctggtttcattaaattttctttagtttgaatttactgttatttggtgtgcatttctatgtttggtgagcggttttctgtgctcagagtgtgtttattttatgtaatttaatttctttgtgagctcctgtttagcaggtcgtcccttcagcaaggacacaaaaagattttaattttatt

SEQ ID NO: 21 - protein, 1046 aa, Artificial Sequence
  Synthetic
MFSFTEAKSKISLWTRSAAPLNNVYLSYSCRCGLGKRKLAGGCCSAPYITCYDSADFRRVQYLYFCLTRYCCLYFFLLLLADWFYKKSSIFFETEFSRGFRTWRKIVKLLYILPKFEMESIMSRGIPSGILEEKAIQFKRAKEGNKPLKDEIPKPEDMYVSHTSKWNVLRKMSQKTVDLSKAAAGMGFINKHMLTGNILAQPTTVLDIPVTKDKTLAMASDFIRKENLKTSAIHIGAIEIIIQSFASPESDLMGGFLLVDSLHTDTANAIRSIFVAPMRGGRPVRVVTFPNTLAPVSCDLNNRFKLICSLPNCDIVQGSQVAEVSVNVAGCATSIEKSHTPSQLYTEEFEKEGAVVVEYLGRQTYCAQPSNLPTEEKLRSLKFDFHVEQPSVLKLSNSCNAHFVKGESLKYSISGKEAENHAVHATVVSREGASAAPKQYDPILGRVLDPRNGNVAFPQMEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQGPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPLLKFRFRDIERSKRSVMVGHTATAA

SEQ ID NO: 22 - protein, 189 aa, Artificial Sequence
  Synthetic
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPL

SEQ ID NO: 23 - protein, 374 aa, Artificial Sequence
  Synthetic
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

(19) United States
(12) Patent Application Publication
(10) Pub. No.: US 2022/0016191 A1
(43) Pub. Date: Jan. 20, 2022

(54) SIMULTANEOUS DELIVERY OF CANCER TREATMENT PROGRAMS TO TUMOR AND IMMUNE CELLS

(71) Applicant: Massachusetts Institute of Technology, Cambridge, MA (US)

(72) Inventors: Ron Weiss, Newton, MA (US); Jin Huh, Watertown, MA (US); Yingzhong Li, Quincy, MA (US)

(73) Assignee: Massachusetts Institute of Technology, Cambridge, MA (US)

(21) Appl. No.: 17/320,965

(22) Filed: May 14, 2021

Related U.S. Application Data
(60) Provisional application No. 63/052,751, filed on Jul. 16, 2020.

(51) Int. Cl. A61K 35/763; C12N 15/86; A61P 35/00

(52) U.S. Cl. CPC A61K 35/763; C12N 15/86; C12N 2710/16632; C12N 2710/16643; A61P 35/00

(57) ABSTRACT

Disclosed herein are genetically modified herpesviruses for the treatment of cancer. Also provided are methods of treating cancer using genetically modified herpesviruses.

### RELATED APPLICATION

This application claims the benefit under 35 U.S.C. § 119(e) of U.S. provisional application No. 63/052,751 filed Jul. 16, 2020 which is incorporated by reference herein in its entirety.

### GOVERNMENT LICENSE RIGHTS

This invention was made with Government support under Grant No. R01 CA206218 and R01 EB025854 awarded by the National Institutes of Health (NIH). The Government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on May 14, 2021, is named M065670516US01-SEQ-NTJ, and is 71,138 bytes in size.

### BACKGROUND

Cancer is a devastating condition caused by aberrant growth of cells and/or the formation of tumors in a subject, interfering with healthy physiology. Anti-tumor immune responses play an important role in the control of cancer. However, the tumor microenvironment is often characterized by immunosuppressive conditions, which hinder the generation of tumor-specific immune responses.

### SUMMARY

Provided herein are populations of herpesviruses for the selective expression of genetic programs in tumor or immune cells for the treatment of cancer. These populations comprise both a replication-competent herpesvirus that expresses its encoded genetic program in tumor cells, and a replication-deficient herpesvirus that express its encoded genetic program in immune cells. Lytic infection by the replication-competent herpesvirus specifically in the tumor microenvironment results causes the death of cancer cells, with release of cellular components stimulating inflammation and recruitment of immune cells. This immunogenic effect, in conjunction with expression of one or more pro-inflammatory cytokines, immunostimulatory ligands, and immune checkpoint inhibitors, counteracts the immunosuppressive environment that often develops in tumors. The replication-deficient herpesvirus, by contrast, infects immune cells, such as CD8+ T cells, to promote an anti-tumor immune response through the expression of an exogenous T cell receptor or chimeric antigen receptor, pro-inflammatory cytokines, and/or checkpoint inhibitors. Additionally, the replication-deficient herpesvirus may redirect infected immune cells to a specific tumor-associated antigen through the expression of an exogenous T cell receptor or chimeric antigen receptor. The combination of an oncolytic virus to modulate the tumor microenvironment and a replication-deficient virus for immune cell reprogramming allow the herpesvirus populations provided herein act synergistically, driving a more effective anti-cancer response than either virus acting independently. Furthermore, both therapeutic viruses being derived from herpesviruses with similar genetics and common requirements for growth permits efficient in vitro production of virus cocktails for administration to a subject.

Accordingly, the present disclosure provides, in some aspects, a herpesvirus population comprising:
(i) a replication-competent herpesvirus comprising a cancer cell state classifier, wherein the cancer cell state classifier comprises:
   (a) a cancer cell sensor circuit comprising a first constitutive promoter operably linked to a nucleic acid encoding:
      (1) one or more target sequences for a first set of cancer input miRNAs; and
      (2) a nucleic acid sequence encoding one or more of a first set of repressors; and
   (b) a cancer cell signal circuit comprising a first subgenomic promoter operably linked to a nucleic acid encoding:
      (1) one or more of a first set of output molecules; and
      (2) a first repressor recognition sequence that is capable of being bound by the first repressor, wherein the first repressor is capable of binding to the first repressor recognition sequence to prevent expression of the first set of output molecules;
(ii) a replication-deficient herpesvirus comprising an immune cell state classifier, wherein the immune cell state classifier comprises:
   (a) an immune cell sensor circuit comprising a second constitutive promoter operably linked to a nucleic acid encoding:
      (1) one or more target sequences for a first set of immune cell input miRNAs; and
      (2) a nucleic acid sequence encoding one or more of a second set of repressors; and
   (b) an immune cell signal circuit comprising a second subgenomic promoter operably linked to a nucleic acid encoding:
      (1) one or more of a second set of output molecules; and
      (2) a second repressor recognition sequence that is capable of being bound by the second repressor, wherein the second repressor is capable of binding to the first repressor recognition sequence to prevent expression of the second set of output molecules.

In some embodiments, the first set of repressors comprises one or more of a first set of repressor RNAi molecules, and the first repressor recognition sequence comprises one or more target sequences for one or more of the first set of repressor RNAi molecules.

In some embodiments, the second set of repressors comprises one or more of a second set of repressor RNAi molecules, and the second repressor recognition sequence comprises one or more target sequences for one or more of the second set of repressor RNAi molecules.

In some embodiments, the first set of repressors comprises one or more of a first set of repressor miRNAs, and the first repressor recognition sequence comprises one or more target sequences for one or more of the first set of repressor miRNAs.

In some embodiments, the second set of repressors comprises one or more of a second set of repressor miRNAs, and the second repressor recognition sequence comprises one or more target sequences for one or more of the second set of repressor miRNAs.

In some embodiments, the first set of repressor miRNAs does not comprise any miRNAs of the second set of repressor miRNAs.

In some embodiments, the second set of repressor miRNAs does not comprise any miRNAs of the first set of repressor miRNAs.

In some embodiments, the first repressor recognition sequence comprises a first endoribonuclease recognition sequence, and the first set of repressors comprises a first endoribonuclease that is capable of cleaving the first endoribonuclease recognition sequence.

In some embodiments, the second repressor recognition sequence comprises a second endoribonuclease recognition sequence, and the second set of repressors comprises a second endoribonuclease that is capable of cleaving the second endoribonuclease recognition sequence.

In some embodiments, the first endoribonuclease is not capable of cleaving the second endoribonuclease ribonuclease recognition sequence

In some embodiments, the second endoribonuclease is not capable of cleaving the first endoribonuclease recognition sequence.

In some embodiments, the first endoribonuclease is a first CRISPR endoribonuclease selected from the group consisting of Cas6, Csy4, CasE, Cse3, LwaCas13a, PspCas13b, RanCas13b, PguCas13b, and RfxCas13d.

In some embodiments, the second endoribonuclease is a second CRISPR endoribonuclease selected from the group consisting of Cas6, Csy4, CasE, Cse3, LwaCas13a, PspCas13b, RanCas13b, PguCas13b, and RfxCas13d.

In some embodiments, the first endoribonuclease and the second endoribonuclease are different endoribonucleases.

In some embodiments, the cancer cell signal circuit further comprises one or more target sequences for a second set of cancer input miRNAs.

In some embodiments, the immune cell signal circuit further comprises one or more target sequences for a second set of immune cell input miRNAs.

In some embodiments, the cancer cell state classifier comprises up to 5 kb, up to 10 kb, up to 15 kb, up to 20 kb, up to 25 kb, up to 30 kb, up to 31 kb, up to 32 kb, up to 33 kb, up to 34 kb, up to 35 kb, up to 36 kb, up to 37 kb, up to 38 kb, up to 39 kb, or up to 50 kb.

In some embodiments, the cancer cell state classifier comprises up to 33 kb.

In some embodiments, the immune cell state classifier comprises up to 5 kb, up to 10 kb, up to 15 kb, up to 20 kb, up to 25 kb, up to 30 kb, up to 31 kb, up to 32 kb, up to 33 kb, up to 34 kb, up to 35 kb, up to 36 kb, up to 37 kb, up to 38 kb, up to 39 kb, or up to 50 kb.

In some embodiments, wherein the immune cell state classifier comprises up to 33 kb.

In some embodiments, the first constitutive promoter is an hEF1a promoter.

In some embodiments, the second constitutive promoter is an hEF1a promoter.

In some embodiments, the cancer cell state classifier is a DNA encoding an RNA replicon, wherein the RNA replicon comprises the cancer cell sensor circuit and the cancer cell signal circuit.

In some embodiments, the immune cell state classifier is a DNA encoding an RNA replicon, wherein the RNA replicon comprises the immune cell sensor circuit and the immune cell signal circuit.

In some embodiments, the cancer cell RNA replicon comprises a nucleic acid sequence encoding one or more proteins that are capable of replicating the cancer cell RNA replicon.

In some embodiments, the immune cell RNA replicon comprises a nucleic acid sequence encoding one or more proteins that are capable of replicating the immune cell RNA replicon.

In some embodiments, one or more of the proteins that are capable of replicating the RNA replicon comprise a destabilization domain.

In some embodiments, the destabilization domain is selected from the group consisting of PEST, a destabilization domain from E. coli dihydrofolate reductase, a destabilization domain derived from human FK506-binding protein (FKBP), and a destabilization domain derived from FKBP-rapamycin-binding (FRB) protein.

In some embodiments, the replication-competent herpesvirus is a herpesvirus selected from the group consisting of herpes simplex virus (HSV)-1, HSV-2, Varicella-Zoster virus (VZV), Epstein-Barr virus (EBV), human cytomegalovirus (CMV), roseolovirus, and Kaposi's sarcoma herpesvirus (KSHV).

In some embodiments, the replication-competent herpesvirus is a herpesvirus selected from the group consisting of herpes simplex virus (HSV)-1, HSV-2, Varicella-Zoster virus (VZV), Epstein-Barr virus (EBV), human cytomegalovirus (CMV), roseolovirus, and Kaposi's sarcoma herpesvirus (KSHV).

In some embodiments, the replication-competent herpesvirus and/or the replication-deficient herpesvirus is HSV-1.

In some embodiments, the replication-competent herpesvirus is HSV-1, and the replication-deficient herpesvirus is HSV-1.

In some embodiments, the first set of output molecules comprises one or more cytokines.

In some embodiments, the second set of output molecules comprises one or more cytokines.

In some embodiments, the first set of output molecules comprises one or more cytokines selected from the group consisting IL-1β, IL-2, IL-4, IL-6, IL-7, IL-10, IL-12, IL-15, IL-17, IL-18, IFN-γ, TNF-α, and GM-CSF.

In some embodiments, the second set of output molecules comprises one or more cytokines selected from the group consisting of IL-1β, IL-2, IL-4, IL-6, IL-7, IL-10, IL-12, IL-15, IL-17, IL-18, IFN-γ, TNF-α, and GM-CSF.

In some embodiments, the first set of output molecules comprises one or more cytokines selected from the group consisting of IL-2, IL-12, and GM-CSF.

In some embodiments, the second set of output molecules comprises one or more cytokines selected from the group consisting of IL-2, IL-12, and GM-CSF.

In some embodiments, the first set of output molecules comprises IL-2, IL-12, and GM-CSF; and the second set of output molecules comprises IL-2, IL-12, and GM-CSF.

In some embodiments, the first set of output molecules comprises one or more antibodies or antigen-binding fragments thereof.

In some embodiments, the second set of output molecules comprises one or more antibodies or antigen-binding fragments thereof.

In some embodiments, the first set of output molecules comprises one or more antibodies selected from the group consisting of an anti-PD-1 antibody, an anti-PD-L1 antibody, and an anti-CTLA-4 antibody, or antigen-binding fragments thereof.

In some embodiments, the second set of output molecules comprises one or more antibodies selected from the group consisting of an anti-PD-1 antibody, an anti-PD-L1 antibody, and an anti-CTLA-4 antibody, or antigen-binding fragments thereof.

In some embodiments, the first set of output molecules comprises an anti-PD-1 antibody or antigen-binding fragment thereof, and an anti-CTLA-4 antibody or antigen-binding fragment thereof.

In some embodiments, the second set of output molecules comprises an anti-PD-1 antibody or an antigen-binding fragment thereof, and an anti-CTLA-4 antibody or an antigen-binding fragment thereof.

In some embodiments, one or more of the antibodies or antigen-binding fragments thereof is a monoclonal antibody, a chimeric antibody, a humanized antibody, a human engineered antibody, a human antibody, a single chain antibody (scFv), or an antibody fragment.

In some embodiments, the second set of output molecules comprises:
(i) a T cell receptor (TCR) alpha chain or portion thereof and/or
(ii) a T cell receptor (TCR) beta chain or portion thereof.

In some embodiments, the TCR alpha chain comprises a TCR alpha variable (TRAV) domain, wherein the TCR beta chain comprises a TCR beta variable (TRBV) domain, wherein covalent or non-covalent bonding of the TCR alpha and beta chains forms an TCR comprising an antigen-binding domain, wherein the antigen-binding domain is capable of binding to an antigen presentation complex, wherein the antigen presentation complex comprises an antigen and an antigen presentation protein.

In some embodiments, the antigen presentation protein is a class I major histocompatibility complex (MHC-I) protein.

In some embodiments, the antigen presentation protein is a class II major histocompatibility complex (MHC-II) protein.

In some embodiments, the second set of output molecules comprises a chimeric antigen receptor (CAR) or portion thereof, wherein the CAR or portion thereof is capable of binding to an antigen.

In some embodiments, the CAR comprises an extracellular single-chain variable fragment (scFv) of an antibody.

In some embodiments, the CAR further comprises a hinge domain, a transmembrane domain, and one or more intracellular signal transduction domains.

In some embodiments, one or more intracellular signal transduction domains are domains of a protein selected from the group consisting of CD28, CD3, and 4-1BB.

In some embodiments, the antigen is a neoantigen and/or a tumor-associated antigen.

In some embodiments, the antigen is a synthetic antigen, wherein the first set of output molecules comprises the synthetic antigen.

In some embodiments, the first set of output molecules comprises a neoantigen and/or a tumor-associated antigen.

In some embodiments, the first set of output molecules comprises a neoantigen and/or a tumor-associated antigen.

In some embodiments, the first set of output molecules comprises one or more immunostimulatory ligands.

In some embodiments, the second set of output molecules comprises one or more immunostimulatory ligands.

In some embodiments, the first set of output molecules comprises one or more immunostimulatory ligands selected from the group consisting of a LysM-containing protein, flagellin-grp170, cowpea mosaic virus (CPMV) small coat protein, and CPMV large coat protein.

In some embodiments, the second set of output molecules comprises one or more immunostimulatory ligands selected from the group consisting of a LysM-containing protein, flagellin-grp170, cowpea mosaic virus (CPMV) small coat protein, and CPMV large coat protein.

In some embodiments, the LysM-containing protein comprises an amino acid sequence with at least 90% sequence identity to the amino acid sequence of SEQ ID NO: 14.

In some embodiments, the flagellin-grp170 comprises an amino acid sequence with at least 90% sequence identity to the amino acid sequence of SEQ ID NO: 19.

In some embodiments, the CPMV small coat protein comprises an amino acid sequence with at least 90% sequence identity to the amino acid sequence of SEQ ID NO: 22.

In some embodiments, the CPMV large coat protein comprises an amino acid sequence with at least 90% sequence identity to the amino acid sequence of SEQ ID NO: 23.

In some embodiments, the LysM-containing protein comprises an amino acid sequence with at least 95% sequence identity to the amino acid sequence of SEQ ID NO: 14.

In some embodiments, the flagellin-grp170 comprises an amino acid sequence with at least 95% sequence identity to the amino acid sequence of SEQ ID NO: 19.

In some embodiments, the CPMV small coat protein comprises an amino acid sequence with at least 95% sequence identity to the amino acid sequence of SEQ ID NO: 22.

In some embodiments, the CPMV large coat protein comprises an amino acid sequence with at least 95% sequence identity to the amino acid sequence of SEQ ID NO: 23.

In some embodiments, the LysM-containing protein comprises the amino acid sequence of SEQ ID NO: 14.

In some embodiments, the flagellin-grp170 comprises the amino acid sequence of SEQ ID NO: 19.

In some embodiments, the CPMV small coat protein comprises the amino acid sequence of SEQ ID NO: 22.

In some embodiments, the CPMV large coat protein comprises the amino acid sequence of SEQ ID NO: 23.

In some aspects, the present disclosure provides a nucleic acid encoding a genome of any of the replication-competent herpesviruses provided herein.

In some aspects, the present disclosure provides a nucleic acid encoding a genome of any of the replication-deficient herpesviruses provided herein.

In some aspects, the present disclosure provides a vector comprising one or more of the nucleic acids encoding a herpesvirus genome provided herein.

In some embodiments, the present disclosure provides a composition comprising one or more of the vectors provided herein.

In some embodiments, the vector is formulated in a lipid nanoparticle.

In some aspects, the present disclosure provides a cell comprising a genome of any of the replication-deficient herpesviruses provided herein.

In some embodiments, the cell is a T cell, T cell precursor, NK cell, or NK cell precursor.

In some embodiments, the cell is a CD4+ T cell.

In some embodiments, the cell is a CD8+ T cell.

In some embodiments, the genome of the replication-deficient herpesvirus is integrated into a chromosome of the cell.

In some aspects, the present disclosure provides a pharmaceutical composition comprising a pharmaceutically acceptable excipient and any one of the herpesvirus populations, nucleic acids encoding a herpesvirus genome, vectors, compositions comprising one or more vectors, or cells provided herein.

In some aspects, the present disclosure provides a method comprising administering to a subject any one of the herpesvirus populations, nucleic acids encoding a herpesvirus genome, vectors, compositions comprising one or more vectors, cells, or pharmaceutical compositions provided herein.

In some embodiments, the subject is a human.

In some embodiments, the subject has or is at risk of developing cancer.

In some embodiments, the cancer is selected from the group consisting of melanoma, squamous cell cancer, small-cell lung cancer, non-small cell lung cancer, lung cell adenocarcinoma, squamous lung cell carcinoma, peritoneal cancer, hepatocellular cancer, gastrointestinal cancer, esophageal cancer, stomach cancer, pancreatic cancer, glioblastoma, cervical cancer, ovarian cancer, liver cancer, bladder cancer, hepatoma, breast cancer, colon cancer, colorectal cancer, endometrial carcinoma, uterine carcinoma, salivary gland carcinoma, kidney cancer, prostate cancer, vulval cancer, thyroid cancer, hepatic carcinoma, gastric cancer, head-and-neck cancer, leukemia, and lymphoma.

### BRIEF DESCRIPTION OF THE DRAWINGS

FIGS. 1A-1B show conceptual diagrams of how multiple genetically modified HSV-1 viruses elicit anti-cancer effects in vivo. FIG. 1A shows that the selective expression of non-replicating HSV-1, but not replication-competent oncolytic HSV-1, in immune cells expresses a TCR, CAR, cytokines, and/or antibodies to prime adaptive immunity to cancer cells, and that infection of tumor cells by replication-competent oncolytic HSV-1 deploys an anti-cancer genetic program. FIG. 1B shows that replication-deficient HSV-1 (Turbo Charger) expresses a CAR and pro-inflammatory factors to prime immune cells for anti-cancer immunity, while oncolytic replication-competent HSV-1 (Tumor Buster) is not activated in immune cells.

FIG. 2 shows an overview of the process for generating a population of HSV-1 viruses for administration to a subject. The process comprises the steps of 1) transfecting the genome of non-replicating HSV-1 into cells, 2) infecting the cells with replicating, oncolytic HSV-1, 3) allowing for the replication of both HSV-1 viruses, and 4) harvesting the produced HSV-1 viruses to prepare a cocktail for injection.

FIG. 3 shows a schematic describing the advantages of using HSV-1 viruses for delivery of genetic programs for the treatment of cancer. Genetically modified HSV-1 viruses can delivery DNA-launched RNA replicons (DREPs) for amplification of gene expression; 2) safety switches enable the selective expression of output molecules in desired cell types and provide a target for on-demand removal of circuits from a system; the large genome size of HSV-1 enables the delivery of large genetic circuits up to 33 kb in length; and genetic circuits can be designed to express one or more output molecules only in cells with a desired miRNA profile characterized by the abundance of some desired miRNAs and the absence of other undesired miRNAs.

FIGS. 4A-4B show the use of a DNA-launched RNA replicon (DREP) for expression of an output molecule, such as the reporter protein mKate, in a DNA concentration-independent manner. FIG. 4A shows the structures of an RNA replicon (sense/positive sense) and its reverse complement (antisense/negative sense), with the negative sense RNA being used as the template for transcription of a subgenomic, positive sense RNA encoding one or more desired output molecules FIG. 4B shows the relative fluorescence of mKate in cells transfected with a construct encoding mKate RNA under the control of a constitutively active CMV promoter (circles), or a construct encoding a DNA-launched RNA replicon that encodes mKate (squares).

FIGS. 5A-5D show the use of positive and negative regulatory factors to enable expression of an output molecule, such as mVenus reporter protein, only in cells that contain a positive signal and do not contain a negative regulator. FIG. 5A shows a schematic outlining positive regulation of mVenus expression using a degradation domain (DDd) upstream of an nsP2 open reading frame, such that mVenus is expressed only in the presence of TMP. FIG. 5B shows data demonstrating that TMP is required for the expression of DREP-encoded mVenus when a DDd is inserted upstream of nsP2. FIG. 5C shows a schematic outline negative regulation of DREP-encoded mVenus by placement of a Csy4 regulation site on the RNA encoded by the DREP. FIG. 5D shows data demonstrating that the presence of Csy4 in cells inhibits the production of DREP-encoded mVenus.

FIGS. 6A-6B show tuning of the relative amounts of expression of multiple output molecules from a DREP. FIG. 6A shows a schematic of a DREP containing multiple open reading frames under the control of different promoters. FIG. 6B shows data demonstrating that the expression of multiple output molecules, such as reporter proteins and chains of an antibody, can be differentially regulated.

FIGS. 7A-7E show schematics describing multiple methods of expressing output molecules from HSV-1 viral vectors, and the effectiveness of each. FIG. 7A shows an overview of a herpesvirus genome engineered to contain a genetic program for expression in tumors or immune cells FIG. 7B shows a schematic of a nucleic acid construct in which a CMV promoter controls expression of an output molecule. FIG. 7C shows a schematic of a nucleic acid construct in which a CMV promoter drives expression of a DREP. FIG. 7D shows the mean number of cells expressing mKate reporter among cells inoculated with herpesviruses encoding the CMV-mKate construct of FIG. 7B (black bars) or the DREP of FIG. 7C (gray bars). FIG. 7E shows the mean number of cells expressing mKate reporter in tumors of mice engrafted with 4T1 tumor cells and inoculated with herpesviruses encoding the CMV-mKate construct of FIG. 7B (black bars) or the DREP of FIG. 7C (gray bars).

FIGS. 8A-8B show the concentrations of human GM-CSF produced in in vitro and in vivo applications of the HSV-1 viruses encoding GM-CSF. FIG. 8A shows the concentration of GM-CSF in cell culture supernatants of cancer cell liens treated with PBS+10% glycerol (black bar), or infected with replication-competent HSV-1 viruses encoding human GM-CSF under the control of a CMV promoter (blue bars), or encoding a DREP encoding human GM-CSF (red bars). FIG. 8B shows the concentration of GM-CSF in tumors of mice engrafted with various cancer cell lines, and administered PBS+10% glycerol (black bars), HSV-1 viruses encoding human GM-CSF under the control of a human CMV promoter (blue bars), or HSV-1 viruses encoding human GM-CSF on a DREP (red bars).

FIGS. 9A-9D show the proportions and abundance of different immune cells in tumors and tumor-draining lymph nodes of mice engrafted with cancer cells and inoculated with PBS+10% glycerol (black bars), HSV-1 viruses encoding human GM-CSF under the control of a CMV promoter (blue bars), or HSV-1 viruses encoding human GM-CSF on a DREP (red bars). FIG. 9A shows the proportions of various immune cells in the tumor-draining lymph node one day after treatment. FIG. 9B shows the proportions of various immune cells in the tumor one day after treatment FIG. 9C shows the number of various immune cells in the tumor-draining lymph node one day after treatment FIG. 9D shows the number of various immune cells in the tumor one day after treatment.

FIGS. 10A-10B show the induction of output molecule, such as mCherry reporter, expression in immune cells by HSV-1 viral vectors administered to mice in which tumor cells had been engrafted. FIG. 10A shows the percentage of each immune cell type that are mCherry+ one day after HSV-1 administration. FIG. 10B shows the number of mCherry+ cells of each immune cell type per milligram of tumor mass one day after HSV-1 administration.

FIG. 11 illustrates two different genetic programs encoded in separate HSV-1 viruses that work synergistically to treat cancer. The replication-deficient (non-replicating) HSV-1 is designed to express a TCR and/or CAR to alter the antigen-specificity of the infected immune cell to target a desired antigen, such as a synthetic antigen displayed on the tumor surface, and express one or more cytokines and/or antibodies to boost the anti-tumor immune response. The replication-competent (replicating) HSV-1 is designed to express one or more cytokines and/or antibodies to counteract the immunosuppressive tumor microenvironment, as well as one or more peptides that can be expressed on MHC-I and targeted by reprogrammed T cells.

### DETAILED DESCRIPTION

Provided herein are populations of herpesviruses for the selective expression of genetic programs in tumor or immune cells for the treatment of cancer. These populations comprise both a replication-competent herpesvirus that expresses its encoded genetic program in tumor cells, and a replication-deficient herpesvirus that express its encoded genetic program in immune cells. Lytic infection by the replication-competent herpesvirus specifically in the tumor microenvironment results causes the death of cancer cells, with release of cellular components stimulating inflammation and recruitment of immune cells. This immunogenic effect, in conjunction with expression of one or more pro-inflammatory cytokines, immunostimulatory ligands, and immune checkpoint inhibitors, counteracts the immunosuppressive environment that often develops in tumors. The replication-deficient herpesvirus, by contrast, primes immune cells, such as CD8+ T cells, for an anti-tumor response through the expression of pro-inflammatory cytokines or checkpoint inhibitors. Additionally, the replication-deficient herpesvirus may redirect infected immune cells to a specific tumor-associated antigen through the expression of an exogenous T cell receptor or chimeric antigen receptor. The combination of an oncolytic virus to modulate the tumor microenvironment and a replication-deficient virus for immune cell reprogramming allow the herpesvirus populations provided herein act synergistically, driving a more effective anti-cancer response than either virus acting independently. Furthermore, both therapeutic viruses being derived from herpesviruses with similar genetics and common requirements for growth permits efficient in vitro production of virus cocktails for administration to a subject.

### Herpesviridae

Aspects of the present disclosure relate to populations of herpesviruses containing genetic programs (cell state classifiers) for the selective expression of one or more output molecules in desired cell types. As used herein, “herpesvirus” and “herpesviruses” refer to viruses of the Herpesviridae family. A detailed overview of Herpesviridae biology is given in chapters 59-65 of Fields Virology (see, e.g., Fields, B. N., Knipe, D. M., Howley, P. M., & Griffin, D. E. (2013). Fields Virology. Philadelphia: Lippincott Williams & Wilkins) and is summarized below for clarity. Herpesviruses are enveloped viruses with large, often complex, double-stranded DNA genomes. Virus particles, also termed “virions,” of enveloped viruses like Herpesviridae comprise an outer lipid membrane, which is generally derived from the cell membrane of a host cell when new virions are released from the host cell by budding. The outer surface of the viral envelope is coated in any proteins that were present on the host cell at the time of budding, such as host cell surface proteins and any viral proteins expressed on the surface of the host cell. Herpesviruses typically express many unique glycoproteins on the surface of infected host cells. The interior of the viral envelope comprises a viral capsid, or protein shell, formed by the ordered arrangement of one or more capsid proteins, or capsomeres. The space between the exterior of the capsid and inner surface of the envelope is referred to as the tegument, comprising viral enzymes. Enzymes of the tegument have multiple functions, including manipulating biochemical processes within the host cell to facilitate viral infection.

The space within the interior of the herpesvirus capsid contains the viral genome. A herpesvirus genome comprises a single linear double-stranded DNA (dsDNA) molecule. Herpesvirus genomes vary in length, from about 120 kb to about 230 kb, and encode from about 60 to 120 genes. As used herein, the length of a genome refers to the number of DNA base pairs that comprise the genome, or the number of nucleotides in one single-stranded (ssDNA) molecule that comprises the dsDNA genome. For example, a herpesvirus genome 120 kb in length comprises two ssDNA molecules that are reverse complements of each other, with each ssDNA molecule comprising 120,000 nucleotides.

Infection of a host cell (target cell) by a herpesvirus begins with attachment of a virus particle to the host cell membrane, such as by binding of a glycoprotein on viral envelope to a structure on the membrane of a host cell. Binding, as used herein, refers to a non-covalent association, such has hydrogen bonding, between two molecules that results in a close proximity between the molecules. These non-covalent interactions may be transient, allowing detachment of one bound molecule from another, or may maintain the association for an extended period.

Following binding of the virion to the host cell membrane, one or more glycoproteins on the surface of the virion undergo a conformational change, such as folding in on themselves (jack-knifing), to place the viral envelope in close proximity to the lipid bilayer of the cell membrane, promoting fusion of the viral envelope with the host cell membrane, which delivers the viral capsid and contents of the viral tegument into the cytoplasm of the cell. The viral capsid, following delivery into the cell, associates with the nuclear membrane of the cell, and delivers the herpesvirus genome into the nucleus of the host cell through nuclear pores.

From the nucleus, the DNA genome of the herpesvirus can be transcribed to produce RNA transcripts encoding viral proteins, which are required to synthesize new virions. Viral polymerases replicate the herpesvirus genome, which are packaged into new virions. Active production of viral proteins, genome replication, and assembly of new herpesvirus particles is referred to as the “lytic cycle” of herpesvirus infection. Herpesviruses may also establish latency in a host cell. A host cell that is latently infected contains a herpesvirus genome, but is not actively producing viral proteins or new virions. The herpesvirus genome may be integrated into a host cell chromosome, or maintained in the nucleus separately from the host genome. In either case, the herpesvirus genome is replicated during mitosis, such that daughter cells contain the herpesvirus genome. A latent herpesvirus genome can be reactivated, either spontaneously or in response to environmental stimuli, such as the presence of one or more cytokines that indicate the cell is in an anti-inflammatory environment suitable for viral replication.

In some embodiments of the populations of herpesviruses provided herein, the population comprises multiple herpesviruses of the same species, or different species of herpesviruses. In some embodiments, the replication-competent and/or the replication-deficient herpesvirus is a herpes simplex virus (HSV)-1. An example of a DNA sequence of an HSV-1 genome is given by Accession No. JX142173. In some embodiments, the replication-competent and/or the replication-deficient herpesvirus is a herpes simplex virus (HSV)-2. An example of a DNA sequence of an HSV-1 genome is given by Accession No. LS480640. In some embodiments, the replication-competent and/or the replication-deficient herpesvirus is a Varicella-Zoster virus (VZV). An example of a DNA sequence of a VZV genome is given by Accession No. X04370. In some embodiments, the replication-competent and/or the replication-deficient herpesvirus is an Epstein-Barr virus (EBV). An example of a DNA sequence of an EBV genome is given by Accession No. V01555. In some embodiments, the replication-competent and/or the replication-deficient herpesvirus is a human cytomegalovirus (CMV). An example of a DNA sequence of a CMV genome is given by Accession No. BK000394. In some embodiments, the replication-competent and/or the replication-deficient herpesvirus is a roseolovirus. An example of a DNA sequence of a roseolovirus genome is given by Accession No. NC_000898. In some embodiments, the replication-competent and/or the replication-deficient herpesvirus is a Kaposi's sarcoma herpesvirus (KSHV). An example of a DNA sequence of a KSHV genome is given by Accession No. NC_009333. In some embodiments, both the replication-competent herpesvirus and the replication-deficient herpesvirus are derived from a herpesvirus selected from the group consisting of HSV-1, HSV-2, VZV, EBV, CMV, roseolovirus and KSHV. In some embodiments, the replication-competent herpesvirus is an HSV-1, and the replication-deficient herpesvirus is an HSV-1.

In some embodiments of the populations of herpesviruses provided herein, the replication-competent herpesvirus is an HSV-1, and the replication-deficient herpesvirus is an HSV-1. HSV-1, or herpes simplex virus 1, is a herpesvirus that infects multiple types of human cells. The HSV-1 genome is approximately 152 kb in length, but longer genomes can be packaged into virus particles, allowing for the production of HSV-1 viruses that encode genetic programs such as one or more of the cell state classifiers and/or output molecules provided herein.

In some embodiments, the population of herpesviruses comprises a replication-competent herpesvirus. As used herein, a “replication-competent herpesvirus” (also referred to herein as a “replicating” herpesvirus) refers to a herpesvirus that is capable of infecting human cells, expressing one or more proteins encoded by the viral genome, and producing new herpesvirus particles (replicating). In some embodiments, the genome of the replication-competent herpesvirus comprises a cell state classifier that promotes replication only in tumor cells or cancerous cells. In some embodiments, a replication-competent herpesvirus is an oncolytic virus. As used herein, an “oncolytic virus” refers to a virus that replicates in, and/or causes the death of, tumor cells or cancerous cells. Tumor cells may be cancer cells or healthy cells present in or near a tumor. A cancer cell is a cell that is able to bypass normal cell cycle control mechanisms. A tumor is a group of multiple cancer cells in a subject, which may also comprise healthy cells and other structures comprising non-cancer cells, such as blood vessels. A replication-competent virus comprising a cancer cell state classifier that causes degradation of the viral genome in cells other than cancer or tumor cells, and thus replicates specifically in cancer or tumor cells, is an oncolytic virus.

In some embodiments, the population of herpesviruses comprises a replication-deficient herpesvirus. As used herein, a “replication-deficient herpesvirus” (also referred to herein as a “non-replicating” herpesvirus) refers to a herpesvirus that is capable of infecting human cells and expressing one or more proteins encoded by the viral genome, but is not capable of producing new herpesvirus particles in a cell that is not infected with a replication-competent herpesvirus. The replication of a replication-competent herpesvirus in a cell that is also infected with a replication-deficient herpesvirus may result in the production of replication-deficient herpesvirus particles, but a replication-deficient herpesvirus is not capable of replicating without the aid of a replication-competent herpesvirus. In some embodiments, the replication-deficient herpesvirus is replication-deficient due to deletion of one or more essential genes from the viral genome, such as the gene encoding infected cell protein (ICP)0 and/or the gene encoding ICP4. Infected cell protein 0, or ICP0, plays multiple roles in viral replication, including counteracting the antiviral activities of interferon. ICP0 is dispensable for replication in cell culture when cells are infected with multiple viral genomes, but essential when cells are infected at low multiplicity of infection. See, e.g., Gu et al. J Virol. 2009. 83(1):181-187. Thus, co-infection with multiple virions, or infection with another virus that does express ICP0, can promote replication of a virus lacking ICP0, but a herpesvirus lacking ICP0 is said to be replication-deficient. Additionally, ICP4 is required for other steps of herpesvirus replication, including genome replication, and ICP4-deficient viral genomes are commonly used as replication-deficient herpesvirus vectors for the production of modified herpesviruses. See, e.g., Marconi et al. Proc Nat Acad Sci USA. 1996. 93 (21):11319-11320.

In some embodiments of the herpesvirus populations provided herein, a herpesvirus encodes a genetic program on a DNA-launched RNA replicon (DREP). An RNA replicon refers to an RNA encoding one or more molecules (e.g., proteins), individually or in conjunction, are capable of replicating the RNA replicon. In some embodiments, the proteins encoded by the RNA replicon are non-structural proteins nsP1, nsP2, nsP3, and nsP4, which form a herpesvirus RNA-dependent RNA polymerase (RdRp), or replicase, that is capable of replicating the RNA replicon. By encoding proteins that are capable of replicating the RNA, an RNA replicon is capable of self-amplification in a cell, provided that the cell can translate the RNA and produce the encoded protein(s). Thus, an RNA replicon may also be referred to as a “self-amplifying RNA.” A single herpesvirus particle or virion, by delivering a DNA genome encoding an RNA replicon to a cell, is therefore capable of producing a large amount of the RNA replicon in a cell, thereby enabling efficient action of the encoded cell state classifier. See, e.g., WO 2020/181058, which is incorporated herein by reference in its entirety.

In some embodiments, one or more of the non-structural proteins of the replicase comprise a destabilization domain. A destabilization domain (DD) is an amino acid sequence that is readily identified and degraded by one or more components of protein quality control machinery within cells of a particular organism, such as, but not limited to, the ubiquitin proteosome system of eukaryotes. A destabilization domain may also be referred to as a “degron” or “degradation domain”. A destabilization domain may be a complete protein or a subset of a protein (e.g., an amino acid sequence corresponding to one or more domains within a protein, or part of a domain thereof).

In some embodiments, one or more proteins encoded by an RNA replicon are fused with a destabilization domain. In such embodiments, fusion with a destabilization domain causes the one or more fused proteins to be targeted by degradation machinery within cells, thereby decreasing their intracellular quantity. In some embodiments, one or more of nsP1, nsP2, nsP3, and nsP4 are fused with a destabilization domain. In such embodiments, the RNA replicon comprises genes encoding these proteins in which a mutation has been made to insert a nucleotide sequence encoding the destabilization domain. Such a mutation is made such that the inserted sequence encoding the destabilization domain is in frame with the nucleotide sequence encoding the protein (e.g., nsP1, nsP2, nsP3, or nsP4), such that when the sequence is transcribed and translated, a fusion protein comprising both the original protein and the destabilization domain is produced. In embodiments where one or more nsPs are fused with a destabilization domain, the RdRp complex consisting of nsP1-4 cannot be efficiently formed. As a result, replication of the RNA replicon and expression of the genes it contains are decreased. The destabilization domain may be stabilized in the presence of a small molecule that interacts directly with the stabilization domain, thereby stabilizing the fused nsP such that functional RdRp complexes are able to be efficiently formed.

In some embodiments, the destabilization domain to be fused with one or more proteins of the RNA replicon (e.g., nsP1, nsP2, nsP3, or nsP4) is a destabilization domain derived from the dihydrofolate reductase (DHFR) of Escherichia coli (DDd). The development of DDds comprising various mutations relative to wild-type E. coli DHFR is well known in the art (see, e.g., Iwamoto et al. (2010). “A general chemical method to regulate protein stability in the mammalian central nervous system” Chem Biol, 17(9), 981-988, which is incorporated by reference herein). Such mutations are well known to, for instance, modify the folding of DHFR and therefore its susceptibility to protein degradation. The small-molecule ligand trimethoprim (TMP) and derivatives thereof stabilize DDd in a rapid, reversible, and dose-dependent manner. For reference, the amino acid sequence of E. coli strain K-12 DHFR comprising R12H and G67S mutations, upon which many DHFR variants are based, is provided in SEQ ID NO: 1.

In some embodiments, the destabilization domain to be fused with one or more proteins of the RNA replicon (e.g., nsP1, nsP2, nsP3, or nsP4) is a destabilization domain derived from the ligand binding domain of human estrogen receptor (DDe). The development of DDes comprising various mutations relative to wild-type human estrogen receptor ligand binding domain is well known in the art (see, e.g., Miyazaki et al. (2012) “Destabilizing domains derived from the human estrogen receptor” J Am Chem Soc, 134(9):3942-5, which is incorporated by reference herein). Such mutations are well known to, for instance, modify the folding of the ligand binding domain and therefore its susceptibility to protein degradation. The small-molecule ligands CMP8 or 4 hydroxytamoxifen (4-OHT) and derivatives thereof stabilize DDe in a rapid, reversible, and dose-dependent manner. For reference, the amino acid sequence of Homo sapiens estrogen receptor ligand binding domain comprising T371A, L384M, M421G, N519S, G521R, and Y537S mutations, upon which many estrogen receptor ligand binding domain variants are based, is provided in SEQ ID NO: 2.

In some embodiments, the destabilization domain to be fused with one or more proteins of the RNA replicon (e.g., nsP1, nsP2, nsP3, or nsP4) is a destabilization domain derived from the human FK506 binding protein (FKBP) (DDf). The development of DDfs comprising various mutations relative to wild-type human FKBP is well known in the art (see, e.g., Banaszynski et al. (2006) “A rapid, reversible, and tunable method to regulate protein function in living cells using synthetic small molecules” Cell, 126(5), 995-1004, which is incorporated by reference herein). Such mutations are well known to, for instance, modify the folding of FKBP and therefore its susceptibility to protein degradation. The small-molecule ligand Shield-1 and derivatives thereof stabilize DDf in a rapid, reversible, and dose-dependent manner. For reference, the amino acid sequence of Homo sapiens FKBP comprising a F36V mutation, upon which many FKBP variants are based, is provided in SEQ ID NO: 3.

In some embodiments, a destabilization domain comprised by one or more proteins encoded by the RNA replicon is stabilized in the presence of one or more molecules, such that the susceptibility of the protein to components of protein degradation machinery is reduced. In some embodiments, the molecule is a small molecule, generally understood in the art to be any molecule with a molecular mass of less than 900 daltons. In some embodiments, a small molecule is cell permeable. In some embodiments, addition of a small molecule to a cell comprising a RNA replicon encoding a fusion protein comprising a destabilization domain causes the intracellular level of the fusion protein to increase relative to the absence of the small molecule. In such embodiments, the intracellular level of the fusion protein may be increased by 10%, increased by 20%, increased by 30%, increased by 40%, increased by 50%, increased by 60%, increased by 70%, increased by 80%, increased by 90%, increased by 100%, increased by 125%, increased by 150%, increased by 175%, increased by 200%, increased by 250%, increased by 300%, increased by 350%, increased by 400%, increased by 450%, increased by 500%, increased by 600%, increased by 700%, increased by 800%, increased by 900%, or increased by 1000% or more.

In some embodiments, the small molecule directly interacts (i.e., binds) with the destabilization domain. In some embodiments, interaction with the small molecule enhances folding of the destabilization domain. In some embodiments, interaction with the small molecule prevents recognition of the destabilization domain by components of protein degradation machinery. In some embodiments, the interaction between the destabilization domain and the small molecule may be characterized in terms of a dissociation constant (KD). In such embodiments, the small molecule interacts with a destabilization domain with a KD of at least 10 pM, at least 20 pM, at least 30 pM, at least 40 pM, at least 50 pM, at least 60 pM, at least 70 pM, at least 80 pM, at least 90 pM, at least 100 pM, at least 125 pM, at least 150 pM, at least 175 pM, at least 200 pM, at least 250 pM, at least 300 pM, at least 350 pM, at least 400 pM, at least 450 pM, at least 500 pM, at least 600 pM, at least 700 pM, at least 800 pM, at least 900 pM, at least 1 nM, at least 10 nM, at least 25 nM, at least 50 nM, at least 75 nM, at least 100 nM, at least 125 nM, at least 150 nM, at least 175 nM, at least 200 nM, at least 250 nM, at least 300 nM, at least 350 nM, at least 400 nM, at least 450 nM, at least 500 nM, at least 600 nM, at least 700 nM, at least 800 nM, at least 900 nM, or at least 1 μM.

In some embodiments where at least one destabilization domain comprised by a fusion protein encoded by the RNA replicon is an E. coli dihydrofolate reductase (DHFR) destabilization domain (DDd), the small molecule is trimethoprim (TMP) or a derivative thereof. Derivatives of trimethoprim are compounds that would generally be understood by those well versed in the art to share structural features of trimethoprim and include, for instance, iodinated trimethoprim (TMP-I) and diaveridine (see, e.g., Nilchan et al. (2018) “Halogenated trimethoprim derivatives as multidrug-resistant Staphylococcus aureus therapeutics” Bioorg Med Chem, 26(19):5343-5348, which is incorporated by reference herein). The use of trimethoprim and derivatives thereof to reduce degradation of proteins containing a DDd is well known in the art, for example, in Iwamoto et al. (2010). “A general chemical method to regulate protein stability in the mammalian central nervous system” Chem Biol, 17(9), 981-988, which is incorporated by reference herein.

In some embodiments where at least one destabilization domain comprised by a fusion protein encoded by the RNA replicon is a human estrogen receptor ligand binding domain destabilization domain (DDe), the small molecule is 4-hydroxytamoxifen (4-OHT) or a derivative thereof. Derivatives of 4-hydroxytamoxifen are compounds that would generally be understood by those well versed in the art to share structural features of 4-hydroxytamoxifen and include, for example, endoxifen (see, e.g., Maximov et al. (2018) “Endoxifen, 4-Hydroxytamoxifen and an Estrogenic Derivative Modulate Estrogen Receptor Complex Mediated Apoptosis in Breast Cancer” Mol Pharmacol, 94(2), 812-822, which is incorporated by reference herein). The use of 4-hydroxytamoxifen and derivatives thereof to reduce degradation of proteins containing a DDe is well known in the art, for example, in Miyazaki et al. (2012) “Destabilizing domains derived from the human estrogen receptor” J Am Chem Soc, 134(9):3942-5, which is incorporated by reference herein.

In some embodiments where at least one destabilization domain comprised by a fusion protein encoded by the RNA replicon is a human FK506 binding protein (FKBP) destabilization domain (DDf), the small molecule is a Shield ligand or a derivative thereof. A Shield ligand may be, for example, Shield-1 or Shield-2 (see, e.g., Grimley et al. (2008) “Synthesis and analysis of stabilizing ligands for FKBP-derived destabilizing domains”. Bioorg Med Chem Lett, 18(2), 759-761, which is incorporated by reference herein), or a derivative compound that would generally be understood by those well versed in the art to share structural features of Shield-1 or Shield-2. The use of Shield ligands to reduce degradation of proteins containing a DDf is well known in the art, for example, in Banaszynski et al. (2006) “A rapid, reversible, and tunable method to regulate protein function in living cells using synthetic small molecules”. Cell, 126(5), 995-1004, which is incorporated by reference herein.

In some embodiments, the destabilization domain is selected from PEST, a destabilization domain from E. coli dihydrofolate reductase, or a destabilization domain derived from human FK506-binding protein (FKBP) or FKBP-rapamycin-binding (FRB) protein. A PEST domain comprises an amino acid sequence that is rich in proline (P), glutamic acid (E), serine (S), and threonine (T). PEST sequences trigger degradation of proteins containing them, and thus proteins containing PEST sequences are less stable in cells that do not have PEST sequences. In the absence of a stabilizing molecule, the protein containing the destabilization domain is readily degraded in cells, preventing it from contributing to replication. Thus, replication of an RNA replicon can be selectively controlled by adding the stabilizing molecule to a system, or prevented by withholding the stabilizing molecule from the system. In some embodiments, the stabilizing molecule is TMP, OHT-1, rapamycin, or a rapalog.

### Cell State Classifiers

Aspects of the present disclosure relate to herpesviruses that selectively express output molecules (e.g., cytokines, antibodies, immunostimulatory ligands, T cell receptors, chimeric antigen receptors) in certain cell types, such as cancer cells, tumor cells, or immune cells. A virus is said to selectively express a gene or protein in a particular cell type if the virus expresses the gene or produces the protein if, and only if, the virus is present in a cell of the particular type. If the virus infects the particular cell type, such as a tumor cell or immune cell, it will express the gene or produce the protein. If the virus infects a cell that is not the particular cell type, then it will not express the gene or produce the protein. Genetic programs, such as cell state classifiers, that enable selective expression allow a virus to be administered to a subject or population of cells containing multiple cell types, and express the encoded output molecules only in a desired cell type.

Replication-competent herpesviruses of the present disclosure contain cancer cell state classifiers comprising a cancer cell sensor circuit and a cancer cell signal circuit. Replication-deficient herpesviruses of the present disclosure contain immune cell state classifiers comprising an immune cell sensor circuit and an immune cell signal circuit. A sensor circuit refers to a nucleic acid comprising features that allow for differential regulation of gene expression depending on whether the sensor circuit is present in a given cell type. A signal circuit refers to a nucleic acid, or group of nucleic acids, encoding one or more output molecules, and comprising features that can be regulated by the activity of the sensor circuit. Sensor circuits of the present disclosure comprise at least one target sequence for a first set of cell-specific input miRNAs, and encode one or more of a set of repressors. Signal circuits of the present disclosure comprise a repressor recognition sequence that is capable of being bound by one of the repressors of the corresponding sensor circuit, with the repressor being capable of binding to the repressor recognition sequence to prevent expression of the set of output molecules encoded by the signal circuit. In the absence of any of the first set of input miRNAs, which serve as a signal that the herpesvirus is present in a desired cell type, the sensor circuit expresses the encoded repressor(s), which bind to the repressor recognition sequence on the signal circuit, preventing expression of the encoded output molecule(s). This negative regulation prevents the herpesvirus from expressing encoded output molecules, such as cytokines, antibodies, and immunostimulatory ligands, in undesired cells. But, if one or more of a desired first set of input miRNAs are present in a cell, the input miRNA will bind to and initiate degradation of the sensor circuit, preventing expression of the repressor(s) and downstream degradation of the signal circuit, allowing expression of the output molecule(s). Thus, the sensor circuit allows the cell state classifier to effectively senses the presence, or high abundance, of one or more of the first set of miRNAs in a cell. Accordingly, the cell state classifier expresses the output molecule(s) encoded on its signal circuit only in cells that are characterized as “miRNA-high” for any one of the first set of input miRNAs. The first set of input miRNAs may contain any combination of miRNAs that are highly abundant in a desired cell type, such as a cancer cell, tumor cell, or immune cell, but not other cells that are not the desired cell type, to allow a cell state classifier to express its encoded output molecule(s) only in cells characterized by high abundance of any of the first set of input miRNAs.

In addition to the mechanism of positive regulation described in the preceding paragraph, in which the presence of a first set of input miRNAs permits expression of output molecules, the signal circuit may also include one or more target sequences for a second set of input miRNAs for negative regulation. Even if one or more of the first set of input miRNAs are present in a cell, causing degradation of the sensor circuit, the presence of one or more of the second set of input miRNAs can cause degradation of the signal circuit, which also inhibits expression of the output molecules. Thus, the signal circuit allows the cell state classifier to effectively senses the absence, or low abundance, of each of the second set of miRNAs in a cell. Accordingly, the cell state classifier expresses the output molecule(s) encoded on its signal circuit only in cells that are characterized as both “miRNA-high” for any one of the first set of input miRNAs, and “miRNA-low” for each of the second set of input miRNAs. The second set of input miRNAs may contain any combination of miRNAs that are absent or present in low amounts in a desired cell type, such as a cancer cell, tumor cell, or immune cell, but not other cells that are not the desired cell type, to allow a cell state classifier to express its encoded output molecule(s) only in cells characterized by the absence or low abundance of any of the second set of input miRNAs.

Replication-competent herpesviruses of the present disclosure comprise a cancer cell state classifier that prevents expression of encoded output molecules when the replication-competent herpesvirus infects cells that are not cancer cells or tumor cells, but permits expression of the output molecules when the replication-competent herpesvirus infects a cancer cell or tumor cell. A cancer cell refers to a cell in which normal regulation of cell grown and/or division has been disrupted, allowing the cell to divide in circumstances that would normally prevent cell division. A tumor refers to a collection of cancer cells, and may include other cells, such as fibroblasts, endothelial cells, and hematopoietic cells, as well as other structures, such as blood vessels. A tumor cell refers a cell present in a tumor, as well as a cell that originated in a tumor and has disseminated to another anatomical site.

In some embodiments of the cancer cell state classifiers provided herein, the cancer cell state classifier comprises a cancer cell sensor circuit and a cancer cell signal circuit. In some embodiments, the cancer cell sensor circuit comprises a first constitutive promoter operably linked to a nucleic acid encoding one or more target sequences for a first set of cancer input miRNAs; and nucleic acid sequence encoding one or more of a first set of repressors. In some embodiments, the cancer cell signal circuit comprises a first subgenomic promoter operably linked to a nucleic acid encoding one or more of a first set of output molecules; and a first repressor recognition sequence that is capable of being bound by one or more first repressors, wherein binding of the first repressor to the first repressor recognition sequence prevents expression of the first set of output molecules. As used herein “subgenomic promoter” refers to a promoter that controls transcription of only some genes in a genome, such as a herpesvirus genome. In some embodiments of the herpesviruses described herein, replication of the herpesvirus, and the RNA replicon, is controlled by an upstream promoter, while a subgenomic promoter controls expression of a sensor circuit, signal circuit, and/or output molecules of the cell state classifier. The RNA replicon can thus be constitutively expressed from the herpesvirus genome, with expression of one or more elements encoded by the RNA replicon being controlled separately through subgenomic promoters.

In some embodiments of the immune cell state classifiers provided herein, the immune cell state classifier comprises an immune cell sensor circuit and an immune cell signal circuit. In some embodiments, the immune cell sensor circuit comprises a second constitutive promoter operably linked to a nucleic acid encoding one or more target sequences for a first set of immune cell input miRNAs; and nucleic acid sequence encoding one or more of a second set of repressors. In some embodiments, the immune cell signal circuit comprises a second subgenomic promoter operably linked to a nucleic acid encoding one or more of a second set of output molecules; and a second repressor recognition sequence that is capable of being bound by one or more second repressors, wherein binding of the second repressor to the second repressor recognition sequence prevents expression of the second set of output molecules.

Replication-deficient herpesvirus of the present disclosure comprise an immune cell state classifier that prevents expression of encoded output molecules when the replication-deficient herpesvirus infects cells that are not immune cells, but permits expression of the output molecules when the replication-competent herpesvirus infects an immune cell. In some embodiments, the immune cell is a T cell or T cell precursor. In some embodiments, the immune cell is a CD4+ T cell, a CD8+ T cell, or an NK-T cell. In some embodiments, the immune cell is a CD8+ T cell.

A promoter is a nucleic acid sequence that controls expression of a gene or nucleic acid sequence to which it is operably linked. A promoter is said to be operably linked to a gene if the promoter controls the degree to which the gene is expressed. A promoter may be a constitutive promoter, which results in expression of an operably linked gene at a consistent level. A promoter may be a conditional promoter, which regulates expression of an operably linked gene based on environmental conditions, such as the presence, absence, or amount of a stimulus, such as a small molecule, protein, or nucleic acid. In some embodiments, the first and/or second constitutive promoters are hEF1a promoters.

A nucleic acid sequence is said to encode a protein or gene product if a nucleic acid comprising the sequence can be translated by cellular machinery, in the case of an RNA sequence, to produce the protein or gene product. If the nucleic acid sequence is a DNA sequence, then it is said to encode a protein or gene product if the DNA sequence can be transcribed to produce an RNA sequence that can then be translated to produce the protein or gene product.

A repressor, as used herein, refers to a protein or nucleic acid molecule that is capable of inhibiting translation of an RNA. In some embodiments of the cell state classifiers provided herein, the repressor is an endoribonuclease, an RNAi molecule, or a ribozyme. RNAi molecules are RNA interference molecules (e.g. microRNA, miRNA, siRNA, shRNA) that bind to RNA molecules with complementary sequences and, following binding, prevent translation and/or induce degradation of the bound RNA molecule. Ribozymes are nucleic acid enzymes, or nucleic acids with catalytic activity. RNAi molecules, ribozymes, and the use of each in silencing gene expression are familiar to those skilled in the art.

In some embodiments, the first and/or second set of repressors comprises a CRISPR endoribonuclease, and the first and/or second repressor recognition sequence comprises a CRISPR endoribonuclease recognition sequence. An endoribonuclease or CRISPR endonuclease, as used herein, refers to a nuclease that cleaves an RNA molecule in a sequence specific manner, e.g., at a target sequence. Sequence-specific endoribonucleases have been described in the art. For example, the Pyrococcus furiosus CRISPR-associated endoribonuclease 6 (Cas6) is found to cleave RNA molecules in a sequence-specific manner (Carte et al., Genes & Dev. 2008. 22: 3489-3496). In another example, endoribonucleases that cleave RNA molecules in a sequence-specific manner are engineered, which recognize an 8-nucleotide (nt) RNA sequence and make a single cleavage in the target (Choudhury et al., Nat Commun 3, 1147 (2012). In some embodiments, the first and/or second endoribonuclease belongs to the CRISPR-associated endoribonuclease family. In some embodiments, the first and/or second endoribonuclease belongs to the CRISPR-associated endoribonuclease 6 (Cas6) family. Cas6 family nucleases from different bacterial species may be used. Non-limiting examples of Cas6 family nucleases include Cas6, Csy4 (also known as Cas6f), Cse3, and CasE. In some embodiments, the first and/or second endoribonuclease is Cas6, Csy4, CasE, Cse3, LwaCas13a, PspCas13b, RanCas13b, PguCas13b, or RfxCas13d. In some embodiments, the first endoribonuclease is not capable of cleaving the second endoribonuclease ribonuclease recognition sequence. In some embodiments, the second endoribonuclease is not capable of cleaving the first endoribonuclease recognition sequence. In some embodiments, the first endoribonuclease and the second endoribonuclease are different endoribonucleases.

A target sequence of an miRNA, as used herein, refers to a nucleic acid sequence that is complementary to an miRNA. A first nucleic acid sequence is complementary to a second nucleic acid sequence if a nucleic acid comprising the first sequence binds to a nucleic acid comprising the second sequence, forming a nucleic acid that is at least partially double-stranded through hydrogen bonds between base pairs on the miRNA and target sequence. A first sequence is most complementary to a second sequence when the first sequence comprises a sequence of bases that form canonical Watson-Crick base pairs (i.e., A-U, A-T, C-G) with the target sequence, in reverse order relative to the order of bases in the target sequence. A nucleic acid with this sequence of complementary bases in reverse order is said to have the reverse complement of the target sequence. For example, the reverse complement of the target sequence AAGUCCA is TGGACTT (DNA) or UGGACUU (RNA). An miRNA may still bind to a target sequence even if the sequence of the miRNA differs from the exact reverse complement of the target sequence by one or more nucleotides, provided the sequence of the miRNA is sufficiently similar to the reverse complement of the target sequence. The exact level of sequence identity between the sequence of an miRNA and the reverse complement of the target sequence that is sufficient for an miRNA to bind to a given target sequence will depend on the sequences of the miRNA and target sequence, for example, the nucleotide composition and/or length, as well as the binding conditions (e.g., in vivo human physiological conditions). Methods of determining whether an miRNA comprising a given sequence binds to a nucleic acid comprising a target sequence are well known in the art. Following binding of an miRNA to a target sequence, the nucleic acid comprising the target sequence is degraded by cellular machinery of the targeted RNA-directed miRNA degradation (TDMD) pathway.

In some embodiments, one or more of the first and/or second set of repressors comprises a repressor miRNA. In some embodiments, the first set of repressors comprises a first set of repressor miRNAs, and the first repressor recognition sequence comprises one or more target sequences for one or more of the first set of repressor miRNAs. In some embodiments, the second set of repressors comprises a second set of repressor miRNAs, and the second repressor recognition sequence comprises one or more target sequences for one or more of the second set of repressor miRNAs. In some embodiments, the second set of repressor miRNAs does not comprise any miRNAs of the first set of repressor miRNAs. In some embodiments, the first set of repressor miRNAs does not comprise any miRNAs of the second set of repressor miRNAs.

In some embodiments, the cancer cell signal circuit further comprises one or more target sequences for a second set of cancer input miRNAs. In some embodiments, the immune cell signal circuit further comprises one or more target sequences for a second set of immune cell input miRNAs. As discussed above, the inclusion of target sequences for a second set of miRNAs allows for the degradation of the signal circuit in the presence of any one of the miRNAs that are capable of binding to a target sequence on the signal circuit.

In some embodiments, the presence or absence of an miRNA is an miRNA biomarker signature for a specific cell type in a specific stage of development, such as an immune cell. In some embodiments, the cell is a T cell precursor. In some embodiments, the cell is a hematopoietic stem cell. In some embodiments, the cell is an immune cell. In some embodiments, the immune cell is a T cell, B cell, NK cell, monocyte, macrophage, dendritic cell, neutrophil, eosinophil, or basophil. In some embodiments, the T cell is a CD4⁺ T cell or a CD8⁺ T cell. In some embodiments, the presence or absence of an miRNA is an miRNA biomarker signature for a diseased cell. Non-limiting examples of a diseased cells are neoplastic cells, infected cells, cells harboring genetic mutations, and fibrogenetic cells. In some embodiments, the presence or absence of an miRNA is an miRNA biomarker signature for a cancer cell or a tumor cell. Methods of identifying an miRNA biomarker signature in a specific tissue or cell are known in the art. Information about the sequences, origins, and functions of known miRNAs maybe found in publicly available databases (e.g., mirbase.org/, all versions, as described in Kozomara et al., Nucleic Acids Res 2014 42:D68-D73; Kozomara et al., Nucleic Acids Res 2011 39:D152-D157; Griffiths-Jones et al., Nucleic Acids Res 2008 36:D154-D158; Griffiths-Jones et al., Nucleic Acids Res 2006 34:D140-D144; and Griffiths-Jones et al., Nucleic Acids Res 2004 32:D109-D111, including the most recently released version miRBase 21, which contains “high confidence” miRNAs).

Non-limiting examples of miRNAs that are expressed in cells and are able to be detected by the cell state classifiers of the present disclosure are: FF4, FF5, hsa-let-7a-2-3p, hsa-let-7a-3p, hsa-let-7a-5p, hsa-let-7b-3p, hsa-let-7b-5p, hsa-let-7c-5p, hsa-let-7d-3p, hsa-let-7d-5p, hsa-let-7e-3p, hsa-let-7e-5p, hsa-let-7f-1-3p, hsa-let-7f-2-3p, hsa-let-7f-5p, hsa-let-7g-3p, hsa-let-7g-5p, hsa-let-7i-5p, hsa-miR-1, hsa-miR-1-3p, hsa-miR-1-5p, hsa-miR-100-3p, hsa-miR-100-5p, hsa-miR-101-3p, hsa-miR-101-5p, hsa-miR-103a-2-5p, hsa-miR-103a-3p, hsa-miR-105-3p, hsa-miR-105-5p, hsa-miR-106a-3p, hsa-miR-106a-5p, hsa-miR-106b-3p, hsa-miR-106b-5p, hsa-miR-107, hsa-miR-10a-3p, hsa-miR-10a-5p, hsa-miR-10b-3p, hsa-miR-10b-5p, hsa-miR-1185-1-3p, hsa-miR-1185-2-3p, hsa-miR-1185-5p, hsa-miR-122-3p, hsa-miR-122a-5p, hsa-miR-1249-3p, hsa-miR-1249-5p, hsa-miR-124a-3p, hsa-miR-125a-3p, hsa-miR-125a-5p, hsa-miR-125b-1-3p, hsa-miR-125b-2-3p, hsa-miR-125b-5p, hsa-miR-126-3p, hsa-miR-126-5p, hsa-miR-127-3p, hsa-miR-1271-3p, hsa-miR-1271-5p, hsa-miR-1278, hsa-miR-128-1-5p, hsa-miR-128-2-5p, hsa-miR-128-3p, hsa-miR-1285-3p, hsa-miR-1285-5p, hsa-miR-1287-3p, hsa-miR-1287-5p, hsa-miR-129-1-3p, hsa-miR-129-2-3p, hsa-miR-129-5p, hsa-miR-1296-3p, hsa-miR-1296-5p, hsa-miR-1304-3p, hsa-miR-1304-5p, hsa-miR-1306-3p, hsa-miR-1306-5p, hsa-miR-1307-3p, hsa-miR-1307-5p, hsa-miR-130a-3p, hsa-miR-130b-3p, hsa-miR-130b-5p, hsa-miR-132-3p, hsa-miR-132-5p, hsa-miR-133a-3p, hsa-miR-133a-5p, hsa-miR-133b, hsa-miR-134-3p, hsa-miR-134-5p, hsa-miR-135a-3p, hsa-miR-135a-5p, hsa-miR-135b-3p, hsa-miR-135b-5p, hsa-miR-136-3p, hsa-miR-136-5p, hsa-miR-138-1-3p, hsa-miR-138-5p, hsa-miR-139-3p, hsa-miR-139-5p, hsa-miR-140-3p, hsa-miR-140-5p, hsa-miR-141-3p, hsa-miR-141-5p, hsa-miR-142-3p, hsa-miR-142-5p, hsa-miR-143-3p, hsa-miR-143-5p, hsa-miR-144-3p, hsa-miR-144-5p, hsa-miR-145-5p, hsa-miR-146a-3p, hsa-miR-146a-5p, hsa-miR-147a, hsa-miR-148a-3p, hsa-miR-148a-5p, hsa-miR-148b-3p, hsa-miR-148b-5p, hsa-miR-149-3p, hsa-miR-144-3p, hsa-miR-150-3p, hsa-miR-150-5p, hsa-miR-151a-3p, hsa-miR-151a-5p, hsa-miR-152-3p, hsa-miR-152-5p, hsa-miR-154-3p, hsa-miR-154-5p, hsa-miR-155-3p, hsa-miR-155-5p, hsa-miR-15a-3p, hsa-miR-15a-5p, hsa-miR-15b-3p, hsa-miR-15b-5p, hsa-miR-16-1-3p, hsa-miR-16-2-3p, hsa-miR-16-5p, hsa-miR-17-3p, hsa-miR-17-5p, hsa-miR-181a-3p, hsa-miR-181a-5p, hsa-miR-181b-2-3p, hsa-miR-181b-5p, hsa-miR-181c-5p, hsa-miR-181d-3p, hsa-miR-181d-5p, hsa-miR-182-3p, hsa-miR-182-5p, hsa-miR-183-3p, hsa-miR-183-5p, hsa-miR-185-3p, hsa-miR-185-5p, hsa-miR-186-3p, hsa-miR-186-5p, hsa-miR-188-3p, hsa-miR-188-5p, hsa-miR-18a-3p, hsa-miR-18a-5p, hsa-miR-18b-5p, hsa-miR-1908-3p, hsa-miR-1908-5p, hsa-miR-190a-3p, hsa-miR-190a-5p, hsa-miR-191-3p, hsa-miR-191-5p, hsa-miR-1910-3p, hsa-miR-1910-5p, hsa-miR-192-3p, hsa-miR-192-5p, hsa-miR-193a-3p, hsa-miR-193a-5p, hsa-miR-193b-3p, hsa-miR-193b-5p, hsa-miR-194-3p, hsa-miR-194-5p, hsa-miR-195-3p, hsa-miR-195-5p, hsa-miR-196a-3p, hsa-miR-196a-5p, hsa-miR-196b-3p, hsa-miR-196b-5p, hsa-miR-197-3p, hsa-miR-197-5p, hsa-miR-199a-3p, hsa-miR-199a-5p, hsa-miR-199b-3p, hsa-miR-199b-5p, hsa-miR-19a-3p, hsa-miR-19a-5p, hsa-miR-19b-1-5p, hsa-miR-19b-2-5p, hsa-miR-19b-3p, hsa-miR-200a-3p, hsa-miR-200a-5p, hsa-miR-200b-3p, hsa-miR-200b-5p, hsa-miR-200c-3p, hsa-miR-200c-5p, hsa-miR-202-3p, hsa-miR-202-5p, hsa-miR-203a-3p, hsa-miR-203a-5p, hsa-miR-204-5p, hsa-miR-208b-3p, hsa-miR-208b-5p, hsa-miR-20a-3p, hsa-miR-20a-5p, hsa-miR-20b-3p, hsa-miR-20b-5p, hsa-miR-21-5p, hsa-miR-210-3p, hsa-miR-210-5p, hsa-miR-211-3p, hsa-miR-211-5p, hsa-miR-2116-3p, hsa-miR-2116-5p, hsa-miR-212-3p, hsa-miR-214-3p, hsa-miR-215-5p, hsa-miR-217, JG miR-218-1-3p, hsa-miR-218-5p, hsa-miR-219a-1-3p, hsa-miR-219a-2-3p, hsa-miR-219a-5p, hsa-miR-219b-3p, hsa-miR-219b-5p, hsa-miR-22-3p, hsa-miR-22-5p, hsa-miR-221-3p, hsa-miR-221-5p, hsa-miR-222-3p, hsa-miR-222-5p, hsa-miR-223-3p, hsa-miR-223-5p, hsa-miR-23a-3p, hsa-miR-23a-5p, hsa-miR-23b-3p, hsa-miR-24-1-5p, hsa-miR-25-3p, hsa-miR-25-5p, hsa-miR-26a-1-3p, hsa-miR-26a-2-3p, hsa-miR-26a-5p, hsa-miR-26b-5p, hsa-miR-27a-3p, hsa-miR-27a-5p, hsa-miR-27b-3p, hsa-miR-27b-5p, hsa-miR-28-3p, hsa-miR-28-5p, hsa-miR-296-3p, hsa-miR-296-5p, hsa-miR-299-3p, hsa-miR-299-5p, hsa-miR-29a-3p, hsa-miR-29a-5p, hsa-miR-29b-1-5p, hsa-miR-29b-3p, hsa-miR-29c-3p, hsa-miR-301a-3p, hsa-miR-301a-5p, hsa-miR-301b-3p, hsa-miR-301b-5p, hsa-miR-302a-3p, hsa-miR-302a-5p, hsa-miR-302b-5p, hsa-miR-302c-3p, hsa-miR-302c-5p, hsa-miR-3065-3p, hsa-miR-3065-5p, hsa-miR-3074-3p, hsa-miR-3074-5p, hsa-miR-30a-3p, hsa-miR-30a-5p, hsa-miR-30b-3p, hsa-miR-30b-5p, hsa-miR-30c-1-3p, hsa-miR-30c-2-3p, hsa-miR-30c-5p, hsa-miR-30d-3p, hsa-miR-30d-5p, hsa-miR-30e-3p, hsa-miR-30e-5p, hsa-miR-31-3p, hsa-miR-31-5p, hsa-miR-3130-3p, hsa-miR-3130-5p, hsa-miR-3140-3p, hsa-miR-3140-5p, hsa-miR-3144-3p, hsa-miR-3144-5p, hsa-miR-3158-3p, hsa-miR-3158-5p, hsa-miR-32-3p, hsa-miR-32-5p, hsa-miR-320a, hsa-miR-323a-3p, hsa-miR-323a-5p, hsa-miR-324-3p, hsa-miR-324-5p, hsa-miR-326, hsa-miR-328-3p, hsa-miR-328-5p, hsa-miR-329-3p, hsa-miR-329-5p, hsa-miR-330-3p, hsa-miR-330-5p, hsa-miR-331-3p, hsa-miR-331-5p, hsa-miR-335-3p, hsa-miR-335-5p, hsa-miR-337-3p, hsa-miR-337-5p, hsa-miR-338-3p, hsa-miR-338-5p, hsa-miR-339-3p, hsa-miR-339-5p, hsa-miR-33a-3p, hsa-miR-33a-5p, hsa-miR-33b-3p, hsa-miR-33b-5p, hsa-miR-340-3p, hsa-miR-340-5p, hsa-miR-342-3p, hsa-miR-342-5p, hsa-miR-345-3p, hsa-miR-345-5p, hsa-miR-34a-3p, hsa-miR-34a-5p, hsa-miR-34b-3p, hsa-miR-34b-5p, hsa-miR-34c-3p, hsa-miR-34c-5p, hsa-miR-3605-3p, hsa-miR-3605-5p, hsa-miR-361-3p, hsa-miR-361-5p, hsa-miR-3613-3p, hsa-miR-3613-5p, hsa-miR-3614-3p, hsa-miR-3614-5p, hsa-miR-362-3p, hsa-miR-362-5p, hsa-miR-363-3p, hsa-miR-363-5p, hsa-miR-365a-3p, hsa-miR-365a-5p, hsa-miR-365b-3p, hsa-miR-365b-5p, hsa-miR-369-3p, hsa-miR-369-5p, hsa-miR-370-3p, hsa-miR-370-5p, hsa-miR-374a-3p, hsa-miR-374a-5p, hsa-miR-374b-3p, hsa-miR-374b-5p, hsa-miR-375, hsa-miR-376a-2-5p, hsa-miR-376a-3p, hsa-miR-376a-5p, hsa-miR-376c-3p, hsa-miR-376c-5p, hsa-miR-377-3p, hsa-miR-377-5p, hsa-miR-378a-3p, hsa-miR-378a-5p, hsa-miR-379-3p, hsa-miR-379-5p, hsa-miR-381-3p, hsa-miR-381-5p, hsa-miR-382-3p, hsa-miR-382-5p, hsa-miR-409-3p, hsa-miR-409-5p, hsa-miR-411-3p, hsa-miR-411-5p, hsa-miR-412-3p, hsa-miR-421, hsa-miR-423-3p, hsa-miR-423-5p, hsa-miR-424-3p, hsa-miR-424-5p, hsa-miR-425-3p, hsa-miR-425-5p, hsa-miR-431-3p, hsa-miR-431-5p, hsa-miR-432-5p, hsa-miR-433-3p, hsa-miR-433-5p, hsa-miR-449a, hsa-miR-449b-5p, hsa-miR-450a-1-3p, hsa-miR-450a-2-3p, hsa-miR-450a-5p, hsa-miR-450b-3p, hsa-miR-450b-5p, hsa-miR-451a, hsa-miR-452-3p, hsa-miR-4524a-3p, hsa-miR-4524a-5p, hsa-miR-4536-3p, hsa-miR-4536-5p, hsa-miR-454-3p, hsa-miR-454-5p, hsa-miR-4707-3p, hsa-miR-4707-5p, hsa-miR-4755-3p, hsa-miR-4755-5p, hsa-miR-4787-3p, hsa-miR-4787-5p, hsa-miR-483-3p, hsa-miR-483-5p, hsa-miR-484, hsa-miR-485-3p, hsa-miR-485-5p, hsa-miR-487b-3p, hsa-miR-487b-5p, hsa-miR-488-3p, hsa-miR-488-5p, hsa-miR-489-3p, hsa-miR-490-3p, hsa-miR-490-5p, hsa-miR-491-3p, hsa-miR-491-5p, hsa-miR-493-3p, hsa-miR-493-5p, hsa-miR-494-3p, hsa-miR-494-5p, hsa-miR-495-3p, hsa-miR-495-5p, hsa-miR-497-3p, hsa-miR-497-5p, hsa-miR-498, hsa-miR-5001-3p, hsa-miR-5001-5p, hsa-miR-500a-3p, hsa-miR-500a-5p, hsa-miR-5010-3p, hsa-miR-5010-5p, hsa-miR-503-3p, hsa-miR-503-5p, hsa-miR-504-3p, hsa-miR-504-5p, hsa-miR-505-3p, hsa-miR-505-5p, hsa-miR-506-3p, hsa-miR-506-5p, hsa-miR-508-3p, hsa-miR-508-5p, hsa-miR-509-3-5p, hsa-miR-509-3p, hsa-miR-509-5p, hsa-miR-510-3p, hsa-miR-510-5p, hsa-miR-512-5p, hsa-miR-513c-3p, hsa-miR-513c-5p, hsa-miR-514a-3p, hsa-miR-514a-5p, hsa-miR-514b-3p, hsa-miR-514b-5p, hsa-miR-516b-5p, hsa-miR-518c-3p, hsa-miR-518f-3p, hsa-miR-5196-3p, hsa-miR-5196-5p, hsa-miR-519a-3p, hsa-miR-519a-5p, hsa-miR-519c-3p, hsa-miR-519e-3p, hsa-miR-520c-3p, hsa-miR-520f-3p, hsa-miR-520g-3p, hsa-miR-520h, hsa-miR-522-3p, hsa-miR-525-5p, hsa-miR-526b-5p, hsa-miR-532-3p, hsa-miR-532-5p, hsa-miR-539-3p, hsa-miR-539-5p, hsa-miR-542-3p, hsa-miR-542-5p, hsa-miR-543, hsa-miR-545-3p, hsa-miR-545-5p, hsa-miR-548a-3p, hsa-miR-548a-5p, hsa-miR-548ar-3p, hsa-miR-548ar-5p, hsa-miR-548b-3p, hsa-miR-548d-3p, hsa-miR-548d-5p, hsa-miR-548e-3p, hsa-miR-548e-5p, hsa-miR-548h-3p, hsa-miR-548h-5p, hsa-miR-548j-3p, hsa-miR-548j-5p, hsa-miR-548o-3p, hsa-miR-548o-5p, hsa-miR-548v, hsa-miR-551b-3p, hsa-miR-551b-5p, hsa-miR-552-3p, hsa-miR-556-3p, hsa-miR-556-5p, hsa-miR-561-3p, hsa-miR-561-5p, hsa-miR-562, hsa-miR-567, hsa-miR-569, hsa-miR-570-3p, hsa-miR-570-5p, hsa-miR-571, hsa-miR-574-3p, hsa-miR-574-5p, hsa-miR-576-3p, hsa-miR-576-5p, hsa-miR-577, hsa-miR-579-3p, hsa-miR-579-5p, hsa-miR-582-3p, hsa-miR-582-5p, hsa-miR-584-3p, hsa-miR-584-5p, hsa-miR-589-3p, hsa-miR-589-5p, hsa-miR-590-3p, hsa-miR-590-5p, hsa-miR-595, hsa-miR-606, hsa-miR-607, hsa-miR-610, hsa-miR-615-3p, hsa-miR-615-5p, hsa-miR-616-3p, hsa-miR-616-5p, hsa-miR-617, hsa-miR-619-5p, hsa-miR-624-3p, hsa-miR-624-5p, hsa-miR-625-3p, hsa-miR-625-5p, hsa-miR-627-3p, hsa-miR-627-5p, hsa-miR-628-3p, hsa-miR-628-5p, hsa-miR-629-3p, hsa-miR-629-5p, hsa-miR-630, hsa-miR-633, hsa-miR-634, hsa-miR-635, hsa-miR-636, hsa-miR-640, hsa-miR-642a-3p, hsa-miR-642a-5p, hsa-miR-643, hsa-miR-645, hsa-miR-648, hsa-miR-6503-3p, hsa-miR-6503-5p, hsa-miR-651-3p, hsa-miR-651-5p, hsa-miR-6511a-3p, hsa-miR-6511a-5p, hsa-miR-652-3p, hsa-miR-652-5p, hsa-miR-653-5p, hsa-miR-654-3p, hsa-miR-654-5p, hsa-miR-657, hsa-miR-659-3p, hsa-miR-660-3p, hsa-miR-660-5p, hsa-miR-664b-3p, hsa-miR-664b-5p, hsa-miR-671-3p, hsa-miR-671-5p, hsa-miR-675-3p, hsa-miR-675-5p, hsa-miR-7-1-3p, hsa-miR-7-5p, hsa-miR-708-3p, hsa-miR-708-5p, hsa-miR-744-3p, hsa-miR-744-5p, hsa-miR-758-3p, hsa-miR-758-5p, hsa-miR-765, hsa-miR-766-3p, hsa-miR-766-5p, hsa-miR-767-3p, hsa-miR-767-5p, hsa-miR-769-3p, hsa-miR-769-5p, hsa-miR-802, hsa-miR-873-3p, hsa-miR-873-5p, hsa-miR-874-3p, hsa-miR-874-5p, hsa-miR-876-3p, hsa-miR-876-5p, hsa-miR-885-3p, hsa-miR-885-5p, hsa-miR-887-3p, hsa-miR-887-5p, hsa-miR-9-3p, hsa-miR-9-5p, hsa-miR-92a-1-5p, hsa-miR-92a-2-5p, hsa-miR-92a-3p, hsa-miR-92b-3p, hsa-miR-92b-5p, hsa-miR-93-3p, hsa-miR-93-5p, hsa-miR-941, hsa-miR-942-3p, hsa-miR-942-5p, hsa-miR-96-3p, hsa-miR-96-5p, hsa-miR-98-3p, hsa-miR-98-5p, hsa-miR-99a-3p, hsa-miR-99a-5p, hsa-miR-99b-3p, and hsa-miR-99b-5p.

A repressor recognition sequence, as used herein, refers to a nucleic acid sequence that is capable of being recognized and bound by a repressor. Binding of a repressor to a repressor recognition sequence refers to association between nucleic acid sequences of the repressor and repressor recognition sequence, if the repressor is an RNAi molecule or ribozyme, or non-covalent association between the repressor and nucleic acid comprising the repressor recognition sequence, if the repressor is an endoribonuclease. If the repressor is an RNAi molecule, the repressor recognition sequence may comprise a nucleic acid sequence that is complementary to a sequence of the RNAi molecule. If the repressor is a ribozyme, the repressor recognition sequence may comprise a nucleic acid sequence that is complementary to a sequence of the RNAi molecule. If the repressor is an endoribonuclease, the repressor recognition sequence may be a nucleic acid sequence that is capable of being cleaved by the endoribonuclease. Repressor recognition sequences that are capable of being cleaved by given endonucleases are known in the art (see, e.g., DiAndreth et al. bioRxiv. 2019. doi: 10.1101/2019.12.15.867150). Following binding of the repressor to a repressor recognition sequence, the bound nucleic acid is degraded by cellular RNA interference machinery, in the case of an RNAi molecule, or cleaved, in the case of a ribozyme or endoribonuclease.

In some embodiments of the herpesvirus populations provided herein, the cancer cell state classifier comprises up to 5 kb, up to 10 kb, up to 15 kb, up to 20 kb, up to 25 kb, up to 30 kb, up to 31 kb, up to 32 kb, up to 33 kb, up to 34 kb, up to 35 kb, up to 36 kb, up to 37 kb, up to 38 kb, up to 39 kb, or up to 50 kb. In some embodiments, the cancer cell state classifier comprises up to 33 kb. In some embodiments, the immune cell state classifier comprises up to 5 kb, up to 10 kb, up to 15 kb, up to 20 kb, up to 25 kb, up to 30 kb, up to 31 kb, up to 32 kb, up to 33 kb, up to 34 kb, up to 35 kb, up to 36 kb, up to 37 kb, up to 38 kb, up to 39 kb, or up to 50 kb. In some embodiments, the immune cell state classifier comprises up to 33 kb.

In some embodiments of the herpesvirus populations provided herein, the cancer cell state classifier is a DNA encoding an RNA replicon, wherein the RNA replicon comprises the cancer cell sensor circuit and the cancer cell signal circuit. An RNA replicon refers to an RNA encoding one or more molecules (e.g., proteins), individually or in conjunction, are capable of replicating the RNA replicon. In some embodiments, the proteins encoded by the RNA replicon are non-structural proteins nsP1, nsP2, nsP3, and nsP4, which form a herpesvirus replicase that is capable of replicating the RNA replicon. By encoding proteins that are capable of replicating the RNA, an RNA replicon is capable of self-amplification in a cell, provided that the cell can translate the RNA and produce the encoded protein(s). Thus, an RNA replicon may also be referred to as a “self-amplifying RNA.” A single herpesvirus particle or virion, by delivering a DNA genome encoding an RNA replicon to a cell, is therefore capable of producing a large amount of the RNA replicon in a cell, thereby enabling efficient action of the encoded cell state classifier. See, e.g., WO 2020/181058.

In some embodiments of the herpesvirus populations provided herein, the immune cell state classifier is a DNA encoding an RNA replicon, wherein the RNA replicon comprises the immune cell sensor circuit and the immune cell signal circuit.

### Output Molecules

Aspects of the present disclosure relate to herpesvirus populations comprising a replication-competent and a replication-deficient herpesvirus, wherein each herpesvirus comprises a cell state classifier encoding one or more output molecules. An output molecule, as used herein, refers to an RNA molecule or protein that is produced only under desired conditions, such as the presence of one or more of a first set of one or more miRNAs, and optionally the absence of one or more of a second set of one or more miRNAs. Non-limiting examples of output molecules include transcription factors, cytokines, chemokines, antibodies, T cell receptors, chimeric antigen receptors, immunostimulatory ligands, neoantigens, tumor-associated antigens, miRNAs, surface markers, cell surface receptors, and Toll-like receptors.

In some embodiments of the herpesvirus populations provided herein, one or both of the herpesviruses encode one or more cytokines as output molecules. Expression of cytokines by any of the herpesviruses provided herein allows for the induction of a desired response by cells of the immune system, such as migration to a tumor, phagocytosis or killing of cancer cells, secretion of pro-inflammatory cytokines, secretion of chemokines to attract other immune cells, migration to a lymph node, antigen presentation, and activation of adaptive immune cells such as B cells and T cells. Cytokines are known in the art, and the term itself refers to a generalized grouping of small proteins that are secreted by certain cells within the immune system and have an effect on other cells. Cytokines are known to enhance the cellular immune response and, as used herein, can include, but are not limited to, TNFα, IFN-γ, IFN-α, TGF-β, IL-1β, IL-2, IL-4, IL-10, IL-13, IL-17, IL-18, and chemokines. Chemokines are useful for studies investigating response to infection, immune responses, inflammation, trauma, sepsis, cancer, and reproduction, among other applications. Chemokines are known in the art, and are a type of cytokines that induce chemotaxis in nearby responsive cells, typically of white blood cells, to sites of infection. Non-limiting examples of chemokines include, CCL14, CCL19, CCL20, CCL21, CCL25, CCL27, CXCL12, CXCL13, CXCL-8, CCL2, CCL3, CCL4, CCL5, CCL11, and CXCL10. Growth factors are known in the art, and the term itself is sometimes interchangeable with the term cytokines. As used herein, the term “growth factors” refers to a naturally occurring substance capable of signaling between cells and stimulating cellular growth. While cytokines may be growth factors, certain types of cytokines may also have an inhibitory effect on cell growth, thus differentiating the two terms. Non-limiting examples of growth factors include Adrenomedullin (AM), Angiopoietin (Ang), Autocrine motility factor, Bone morphogenetic proteins (BMPs), Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGF5), Fibroblast growth factor 6 (FGF6), Fibroblast growth factor 7 (FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Fetal Bovine Somatotrophin (FBS), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukin-1 (IL-1), IL-2, IL-3, IL-4, IL-5, IL-6, IL-7, Keratinocyte growth factor (KGF), Migration-stimulating factor (MSF), Macrophage-stimulating protein (MSP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-β), Tumor necrosis factor-alpha (TNF-α), and Vascular endothelial growth factor (VEGF).

In some embodiments, the replication-competent herpesvirus genome comprises one or more nucleic acid sequences encoding one or more cytokines selected from the group consisting of IL-1β, IL-2, IL-4, IL-6, IL-7, IL-10, IL-12, IL-15, IL-17, IL-18, IFN-γ, TNF-α, and GM-CS. In some embodiments, the replication-competent herpesvirus genome comprises one or more nucleic acid sequences encoding IL-2, IL-12, and/or GM-CSF. In some embodiments, the replication-competent herpesvirus genome comprises one or more nucleic acid sequences encoding IL-2, IL-12, and GM-CSF. An example of a DNA sequence encoding human IL-2 is given by Accession No. DQ861285, and reproduced below as SEQ ID NO: 4. An example of an amino acid sequence of human IL-2 is given by Accession No. Q0GK43, and reproduced below as SEQ ID NO: 5. Human IL-12 comprises a dimer of two proteins, human IL-12 p35 and human IL-12 p40. An example of a DNA sequence encoding human IL-12 p35 is given by Accession No. AAK84425, and reproduced below as SEQ ID NO: 6. An example of an amino acid sequence of human IL-12 p35 is given by Accession No. P29459, and reproduced below as SEQ ID NO: 7. An example of a DNA sequence encoding human IL-12 p40 is given by Accession No. AF180563, and reproduced below as SEQ ID NO: 8. An example of an amino acid sequence of human IL-12 p40 is given by Accession No. P29460, and reproduced below as SEQ ID NO: 9. An example of a DNA sequence encoding human GM-CSF is given by Accession No. M11734, and reproduced below as SEQ ID NO: 10. An example of an amino acid sequence of human GM-CSF is given by Accession No. P04141, and reproduced below as SEQ ID NO: 11.

In some embodiments, the replication-deficient herpesvirus genome comprises one or more nucleic acid sequences encoding one or more cytokines selected from the group consisting of IL-1β, IL-2, IL-4, IL-6, IL-7, IL-10, IL-12, IL-15, IL-17, IL-18, IFN-γ, TNF-α, and GM-CSF. In some embodiments, the replication-deficient herpesvirus genome comprises one or more nucleic acid sequences encoding IL-2, IL-12, and/or GM-CSF. In some embodiments, the replication-deficient herpesvirus genome comprises one or more nucleic acid sequences encoding IL-2, IL-12, and GM-CSF. In some embodiments of the herpesvirus populations provided herein, one or both of the herpesviruses encodes an antibody or antigen-binding fragment thereof as an output molecule. Antibodies or antigen-binding fragments encoded by herpesviruses of the present disclosure allow the neutralization or inhibition of an undesired molecule, such as an immune checkpoint. As used herein, “immune checkpoint” refers to a molecule (e.g, protein receptor or fragment of a receptor) that inhibits the activity of an immune cell (e.g., a T cell). Non-limiting examples of immune checkpoint inhibitors include PD-1, PD-L1, and CTLA-4. Programmed cell death protein 1 (PD-1) is expressed by T cells and other cells, and is stimulated by interaction with PD-L1. Stimulation of PD-1 inhibits the function of PD-1+ T cells, such as killing of infected cells or cytokine secretion by CD8+ T cells, and thus PD-1 signaling can limit anti-tumor immunity. PD-L1 is a receptor expressed by many cell types, and is able to stimulate PD-1, thereby inhibiting the activity of PD-1+ immune cells such as T cells. Inhibiting stimulation of PD-1 through the use of anti-PD-1 antibodies and/or anti-PD-L1 antibodies is thus useful for enhancing anti-tumor immunity in a subject. CTLA-4 is another immune checkpoint expressed on immune cells, such as T cells, that acts as a regulator of T cell activity, and CTLA-4 stimulation can limit the anti-tumor activities of T cells. Similarly, inhibiting CTLA-4 stimulation through the use of anti-CTLA-4 antibodies is useful for enhancing the anti-tumor activities of T cells in a subject. In some embodiments, the first set of output molecules encoded by the replication-competent herpesvirus comprises one or more antibodies or antigen-binding fragments thereof. In some embodiments, the first set of output molecules encoded by the replication-competent herpesvirus comprises one or more antibodies selected from the group consisting of an anti-PD-1 antibody, an anti-PD-L1 antibody, and an anti-CTLA-4 antibody, or antigen-binding fragments thereof. In some embodiments, the second set of output molecules encoded by the replication-deficient herpesvirus comprises one or more antibodies or antigen-binding fragments thereof. In some embodiments, the second set of output molecules encoded by the replication-deficient herpesvirus comprises one or more antibodies selected from the group consisting of an anti-PD-1 antibody, an anti-PD-L1 antibody, and an anti-CTLA-4 antibody, or antigen-binding fragments thereof. In some embodiments, one or more of the antibodies or antigen-binding fragments thereof is a monoclonal antibody, a chimeric antibody, a humanized antibody, a human engineered antibody, a human antibody, a single chain antibody (scFv), or an antibody fragment.

As used herein, “antibody” refers to a polypeptide of the immunoglobulin family that is capable of binding a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies include, but are not limited to, monoclonal antibodies, human antibodies, humanized antibodies, camelid antibodies, chimeric antibodies, and anti-idiotypic (anti-Id) antibodies (including, e.g., anti-Id antibodies to antibodies of the present disclosure). The antibodies can be of any isotype/class (e.g., IgG, IgE, IgM, IgD, IgA and IgY), or subclass (e.g., IgG1, IgG2, IgG3, IgG4, IgA1 and IgA2).

As used herein, “complementarity-determining domains” or “complementary-determining regions” (“CDRs”) interchangeably refer to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting about 15-20% of the variable domains. CDRs can be referred to by their region and order. For example, “VHCDR1” or “HCDR1” both refer to the first CDR of the heavy chain variable region. The CDRs are structurally complementary to the epitope of the target protein and are thus directly responsible for the binding specificity. The remaining stretches of the VL or VH, the so-called framework regions, exhibit less variation in amino acid sequence (Kuby, Immunology, 4th ed., Chapter 4. W.H. Freeman & Co., New York, 2000).

The positions of the CDRs and framework regions can be determined using various well known definitions in the art, e.g., Kabat, Chothia, and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HC CDR1), 50-65 (HC CDR2), and 95-102 (HC CDR3) in a VH, e.g., a mammalian VH, e.g., a human VH; and amino acid residues 24-34 (LC CDR1), 50-56 (LC CDR2), and 89-97 (LC CDR3) in a VL, e.g., a mammalian VL, e.g., a human VL.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively.

As used herein, “antigen binding fragment” refers to one or more portions of an antibody that retain the ability to specifically interact with (e.g., by binding, steric hindrance, stabilizing/destabilizing, spatial distribution) an epitope of an antigen. Examples of binding fragments include, but are not limited to, single-chain Fvs (scFv), disulfide-linked Fvs (sdFv), Fab fragments, F(ab′) fragments, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., Nature 341:544-546, 1989), which consists of a VH domain; and an isolated complementarity determining region (CDR), or other epitope-binding fragments of an antibody.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (“scFv”); see, e.g., Bird et al., Science 242:423-426, 1988; and Huston et al., Proc. Natl. Acad. Sci. 85:5879-5883, 1988). Such single chain antibodies are also intended to be encompassed within the term “antigen-binding fragment.” These antigen binding fragments are obtained using conventional techniques known to those of skill in the art, and the fragments are screened for utility in the same manner as are intact antibodies.

Antigen-binding fragments can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can be grafted into scaffolds based on polypeptides such as fibronectin type III (Fn3) (see U.S. Pat. No. 6,703,199, which describes fibronectin polypeptide monobodies).

Antigen-binding fragments can be incorporated into single chain molecules comprising a pair of tandem Fv segments (VH-CH1-VH-CH1) which, together with complementary light chain polypeptides, form a pair of antigen binding regions (Zapata et al., Protein Eng. 8:1057-1062, 1995; and U.S. Pat. No. 5,641,870).

In some embodiments, the antibody is a monoclonal antibody or antigen-binding fragment thereof. The term “monoclonal antibody” refers to proteins or polypeptides derived from the same genetic source, and thus having substantially identical amino acid sequences.

In some embodiments, the antibody is a chimeric antibody or antigen-binding fragment thereof. A chimeric antibody is an antibody comprising amino acid sequences from different genetic sources. In some embodiments, the chimeric antibody comprises amino acid sequences from a mouse and amino acid sequences from a human. In some embodiments a chimeric antibody comprises a variable domain derived from a mouse and constant domains derived from a human.

In some embodiments, the antibody is a humanized antibody or antigen-binding fragment thereof. A humanized antibody is an antibody comprising constant domains and framework regions derived from a human. In some embodiments, a humanized antibody comprises one or more CDRs derived from a non-human animal. Non-limiting examples of non-human animals from which CDRs may be derived include mice, rats, hamsters, rabbits, goats, sheep, and non-human primates.

In some embodiments, the antibody is a human engineered antibody. A human engineered antibody refers to an antibody derived from a non-human source, such as mouse, in which one or more substitutions have been made to improve a desired characteristic of the antibody, such as to increase stability or reduce immunogenicity when the antibody is administered to a subject. In some embodiments, the substitutions are made at low-risk positions (e.g. exposed to solvent but not contributing to antigen binding or antibody structure). Such substitutions mitigate the risk that a subject will generate an immune response against the antibody following its administration, without affecting the ability of the antibody to bind to a desired epitope or antigen (see, e.g. Studnicka et al. Protein Eng. 1994. 7(6):805-814).

In some embodiments, the antibody is a single chain antibody or antigen-binding fragment. A single chain antibody, or single chain variable fragment (scFV) is a protein or polypeptide comprising a VH domain and a VL domain joined together, such as by a synthetic linker, to form a single protein or polypeptide (see, e.g., Bird et al., Science. 242:423-426, 1988; and Huston et al., Proc. Natl. Acad. Sci. 85:5879-5883, 1988).

In some embodiments, the antibody is an antibody fragment or antigen-binding fragment. An antibody fragment is protein or polypeptide derived from an antibody. An antigen-binding fragment is a protein or polypeptide derived from an antibody that is capable of binding to the same epitope or antigen as the antibody from which it was derived.

In some embodiments of the herpesvirus populations provided herein, one or both of the herpesviruses encodes an immunostimulatory ligand or antigen as an output molecule. As used herein, “immunostimulatory ligand” refers to a molecule that activates a response by a cell of the immune system. Non-limiting examples of responses elicited by immunostimulatory ligands include cytokine secretion, chemokine secretion, migration, phagocytosis, release of cytotoxic granules, upregulation of antigen presentation, and expression of co-stimulatory ligands such as CD80, CD86, CD40, ICOSL, CD70, OX40L, 4-1BBL, GITRL, LIGHT, TIM3, TIM4, ICAM-1, and LFA-3 (see, e.g., Hubo et al. Front Immunol. 2013. 4:82). Generally, the presence of an immunostimulatory ligand elicits an inflammatory response that promotes migration and activation of innate immune cells, such as monocytes, macrophages, dendritic cells, and neutrophils. Activated innate immune cells, such as activated dendritic cells, express pro-inflammatory cytokines, such as IL-1β and IL-18, and are more effective at presenting antigens to T cells. The presence of an immunostimulatory ligand in the body of a subject increases the number of activated immune cells, such as dendritic cells, and promotes their migration to lymph nodes, where antigen presentation activates adaptive immune cells, such as CD4+ T cells, CD8+ T cells, and NK-T cells. Expression of an immunostimulatory ligand by an immune cell, such as an immune cell infected with one of the replication-deficient herpesviruses provided herein, is expected to enhance the generation of an anti-cancer adaptive immune response. When expressed in a tumor, such as by any of the replication-competent herpesviruses provided herein, the presence of an immunostimulatory ligand can activate immune cells within the tumor, counteracting the immunosuppressive environment of the tumor.

In some embodiments, the immunostimulatory ligand is a LysM-containing protein. In some embodiments, the LysM domain-containing protein is a Listeria monocytogenes p60 protein or a fragment thereof. The Listeria monocytogenes p60 protein, particularly the N-terminal domain, enhances the ability of dendritic cells to activate NK cells and stimulate IFN-γ secretion, which play important roles in anti-cancer immunity (see, e.g., Schmidt et al. PLoS Pathog. 2011. 7(11):e1002368). An example of a DNA sequence encoding L. monocytogenes p60 is given by Accession No. AF532267, and reproduced below as SEQ ID NO:12. An example of an amino acid sequence of L. monocytogenes p60 is given by Accession No. Q83TQ3, and reproduced below as SEQ ID NO: 13. In some embodiments the LysM-containing protein comprises an amino acid sequence with at least 90%, at least 91%, at least 92%, at least 93%, at least 94%, at least 95%, at least 96%, at least 97%, at least 98%, at least 99%, or up to 100% of the amino acid sequence of SEQ ID NO: 14. In some embodiments, the LysM-containing protein comprises an amino acid sequence with at least 95% of the amino acid sequence of SEQ ID NO: 14. In some embodiments, the LysM-containing protein comprises the amino acid sequence of SEQ ID NO: 14.

In some embodiments, the immunostimulatory ligand is flagellin-grp170. Flagellin-grp170 is a protein comprising an amino acid sequence derived from bacterial flagellin; and an amino acid sequence derived from grp170. Flagellin is a protein component of bacterial flagella, and many vertebrates, including humans, express receptors that upregulate multiple immune responses, including the activation of dendritic cells, following detection of flagellin. A flagellin-grp170 protein induces multiple responses that enhance antitumor immunity, including elevated expression of IFN-γ and IL-12, and increased infiltration of CD8+ T cells into the tumor microenvironment (see, e.g., Yu et al. Cancer Res. 2013. 73(7):2093-2103). An example of a DNA sequence encoding Salmonella enterica flagellin is given by Accession No. AAL20871, and reproduced below as SEQ ID NO: 15. An example of an amino acid sequence of Salmonella enterica flagellin is given by Accession No. P06179, and reproduced below as SEQ ID NO: 16. An example of a DNA sequence encoding grp170 is given by Accession No. AF228709, and reproduced below as SEQ ID NO: 17. An example of an amino acid sequence of grp170 is given by Accession No. Q9Y4L1, and reproduced below as SEQ ID NO: 18. In some embodiments the flagellin-grp170 comprises an amino acid sequence with at least 90%, at least 91%, at least 92%, at least 93%, at least 94%, at least 95%, at least 96%, at least 97%, at least 98%, at least 99%, or up to 100% of the amino acid sequence of SEQ ID NO: 19. In some embodiments, the flagellin-grp170 protein comprises an amino acid sequence with at least 95% of the amino acid sequence of SEQ ID NO: 19. In some embodiments, the flagellin-grp170 comprises the amino acid sequence of SEQ ID NO: 19.

In some embodiments, the immunostimulatory ligand is a cowpea mosaic virus (CPMV) coat protein. The CPMV capsid comprises a structure formed by multiple copies of large (CPMV-L) and small (CPMV-S) capsid proteins. Virus-like particles containing these cowpea mosaic virus proteins have been shown to enhance antitumor immunity in several cancer models, through the induction of IL-12 IFN-γ expression and enhanced activation of adaptive immune cells (see, e.g., Lizotte et al. Nat Nanotechnol. 2016. 11(3):295-303). Both capsid proteins are formed by translation of a single polypeptide, referred to as M, VP60, or RNA2 polypeptide, followed by cleavage of the polypeptide to release individual proteins, which include CMPV-L, CPMV-S, and other proteins. In some embodiments, the CPMV coat protein is a lar ge (CPMV-L) coat protein. In some embodiments, the CPMV coat protein is a small (CPMV-S) coat protein. An example of a DNA sequence encoding an RNA2 polypeptide is given by Accession No. X00729, and reproduced below as SEQ ID NO: 20. An example of an amino acid sequence of CPMV RNA2 polypeptide is given by Accession No. P03599, and reproduced below as SEQ ID NO: 21. An example of an amino acid sequence of CPMV-S is given by amino acids 834-1022 of Accession No. P03599, and reproduced below as SEQ ID NO: 22. An example of an amino acid sequence of CPMV-L is given by amino acids 460-833 of Accession No. P03599, and reproduced below as SEQ ID NO: 23. In some embodiments the CPMV-S protein comprises an amino acid sequence with at least 90%, at least 91%, at least 92%, at least 93%, at least 94%, at least 95%, at least 96%, at least 97%, at least 98%, at least 99%, or up to 100% of the amino acid sequence of SEQ ID NO: 22. In some embodiments, the CPMV-S protein comprises an amino acid sequence with at least 95% of the amino acid sequence of SEQ ID NO: 22. In some embodiments, the CPMV-S protein comprises the amino acid sequence of SEQ ID NO: 22. In some embodiments the CPMV-L protein comprises an amino acid sequence with at least 90%, at least 91%, at least 92%, at least 93%, at least 94%, at least 95%, at least 96%, at least 97%, at least 98%, at least 99%, or up to 100% of the amino acid sequence of SEQ ID NO: 23. In some embodiments, the CPMV-L protein comprises an amino acid sequence with at least 95% of the amino acid sequence of SEQ ID NO: 23. In some embodiments, the CPMV-L protein comprises the amino acid sequence of SEQ ID NO: 23.

Sequence identity can be determined using the methods described herein, for example, aligning two sequences using BLAST, ALIGN, CLUSTAL, CLUSTALW, or another alignment software or algorithm known in the art. Percent (%) sequence identity, with respect to a reference amino acid sequence, refers to the percentage of amino acids in a candidate sequence that are identical to the amino acids in the reference amino acid sequence, after aligning the sequences and introducing gaps, if necessary, to achieve maximum percent sequence identity. The percent sequence identity between a candidate amino acid sequence (A) and amino acid sequence (B) can be calculated as follows: % sequence identity=100 times X/Y, where X is the number of amino acids in A that are identical to the amino acids in the corresponding positions in B, after alignment, and Y is the total number of amino acids in B. In some embodiments, the amino acid sequence comprises at least 90%, at least 91%, at least 92%, at least 93%, at least 94%, at least 95%, at least 96%, at least 97%, at least 98%, or at least 99% sequence identity to a reference sequence.

In some embodiments, the first set of output molecules encoded by the replication-competent herpesviruses comprises a neoantigen and/or tumor-associated antigen. Expression of a neoantigen or tumor-associated antigen by the replication-competent herpesvirus in the tumor microenvironment can facilitate activation of immune cells, such as T cells, specific to the expressed neoantigen and/or tumor-associated antigen, thereby enhancing the anti-tumor immune response.

In some embodiments, the replication-competent herpesvirus encodes a synthetic antigen. As used herein, a “synthetic antigen” refers to an antigen that is not encoded by the human genome. A synthetic antigen would thus be recognized as a non-self antigen when expressed in a human body, eliciting an immune response when the replication-competent herpesvirus expresses the synthetic antigen in a human.

In some embodiments of the herpesvirus populations provided herein, the replication-deficient herpesvirus encodes a T cell receptor as an output molecule. Expression of a T cell receptor by an immune cell infected with a replication-deficient herpesvirus allows the herpesvirus to control the specificity of the infected immune cell, such that the infected immune cell is activated when the encoded T cell receptor recognizes its target antigen. For example, the T cell receptor encoded by the replication-deficient herpesvirus may be specific to a neoantigen or tumor-associated antigen, and thus the immune cell expressing the T cell receptor will be activated by cells of the tumor and. If the cell is a cytotoxic cell, such as a CD8+ T cell, NK cell, or NK-T cell, the cell will kill cells expressing the target antigen, such as cancer cells.

The T-cell receptor (TCR) is a molecule (e.g., protein) found on the surface of T-cells (i.e., a type of lymphocytes, formed in the thymus which expresses the TCR) which is responsible for recognizing fragments of antigen as peptides bound to major histocompatibility complex (MHC) molecules. The TCR comprises an αβ (alpha-beta) antigen sensing subunit (distinct to each T-cell and having an alpha chain and a beta chain), which is non-covalently linked to the signaling subunit, collectively known as CD3 signaling complex (the CD3εγ (CD3epsilon-CD3gamma) heterodimer, CD3εδ (CD3epsilon-CD3delta) heterodimer, and the CD3ζζ (CD3zeta-CD3zeta) homodimer, each of which may be referred to herein as a CD3 signaling subunit of the CD3 signaling complex).

When the TCR engages with the MHC, through presentation of an antigen to the TCR, the TCR recognizes the antigen and initiates signal transduction. This signal transduction occurs through the various components of the signaling domains (e.g., CD3 signaling subunits) associated with the TCR, which form the TCR complex. This signaling can occur through a variety of mechanisms known in the art, for example by a series of biochemical events mediated by associated enzymes, co-receptors, specialized adaptor molecules, and activated or released transcription factors.

TCRs of the disclosure comprise an alpha chain and a beta chain. In some embodiments the amino acid sequence that corresponds to a naturally occurring amino acid sequence, has at least 96%, 97%, 98%, 99%, 99.5%, 99.9% or more sequence identity to the naturally occurring amino acid sequence. A nucleic acid sequence that corresponds to a naturally occurring nucleic acid sequence of a TCR, has at least 80% sequence identity to the naturally occurring nucleic acid sequence. In some embodiments the nucleic acid sequence that corresponds to a naturally occurring nucleic acid sequence of a TCR, has at least 85%, 88%, 89%, 90%, 91%, 92%, 93%, 94%, 95%, 96%, 97%, 98%, 99%, 99.5%, 99.9% or more sequence identity to the naturally occurring nucleic acid sequence.

In a TCR, a TCR alpha chain is typically linked through a disulfide bond to a beta chain. Each of the alpha and beta chains are comprised of a variable and a constant region. The constant region is proximal to the cell membrane and includes a transmembrane region and a short cytoplasmic tail. The variable region is extracellular and includes the region that binds to a peptide/MHC complex. The variable region includes three complementarity determining regions (CDRs), that provide specificity to the TCR. Each of the alpha and beta chains has at least three CDRs. The CDR3 region is mainly responsible for recognizing the processed antigen. CDR1 of the alpha chain in some instances interacts with the N-terminal portion of the peptide and CDR1 of the beta chain may interact with the C-terminal portion of the peptide. CDR2 may recognize and interact with the MHC.

In some embodiments the TCR includes an alpha chain. In some embodiments the TCR includes a region that is a portion of an alpha chain. A portion of an alpha chain is, for instance, one or more CDRs. In some embodiments an TCR may have an alpha chain CDR3, CDR2, and/or CDR1 or combinations thereof. In some embodiments the TCR includes a beta chain. In other embodiments the TCR includes a region that is a portion of a beta chain. A portion of a beta chain is, for instance, one or more CDRs. In some embodiments, a TCR may have a beta chain CDR3, CDR2, and/or CDR1 or combinations thereof.

The endogenous CDR of an TCR corresponds to the CDR region of a naturally occurring TCR. An amino acid sequence that corresponds to a naturally occurring amino acid sequence of a CDR, has at least 95% sequence identity to the naturally occurring amino acid sequence of a CDR. In some embodiments the amino acid sequence that corresponds to a naturally occurring amino acid sequence of a CDR has at least 96%, 97%, 98%, 99%, 99.5%, 99.9% or more sequence identity to the naturally occurring amino acid sequence of the CDR. A nucleic acid sequence that corresponds to a naturally occurring nucleic acid sequence of a CDR of a TCR, has at least 80% sequence identity to the naturally occurring nucleic acid sequence of the CDR. In some embodiments the nucleic acid sequence that corresponds to a naturally occurring nucleic acid sequence of a CDR of a TCR, has at least 85%, 86%, 87%, 88%, 89%, 90%, 91%, 92%, 93%, 94%, 95%, 96%, 97%, 98%, 99%, 99.5%, 99.9% or more sequence identity to the naturally occurring nucleic acid sequence of the CDR.

In some embodiments the TCR comprises an intracellular domain (ICD) or a portion thereof. For instance, the TCR may comprise one or more co-stimulatory domains, inhibitory domains, signaling components (e.g., signaling components related or involved in T-cell persistence, for example, IL-2 complex (or pieces thereof) or related molecules). As used herein, a “domain” refers to a region in a polypeptide which is independent of other regions in the polypeptide, either functionally or structurally. The terms “intracellular domain,” “intracellular signaling domain,” or “ICD,” may be used interchangeably herein, to mean any domain, oligopeptide, polypeptide, or group of domains, which are internal to the membrane of a cell which may function to facilitate the signal of a TCR or TCR complex subsequent to antigen binding. The ICD, transmits a signal to cause activation or inhibition of a biological process in a cell (e.g., signaling components, co-stimulatory signals, inhibitory signals, or other signaling components).

A portion of an ICD domain is a part of a typical signaling domain or ICD. It may be linked to a whole or partial ICD or an alpha, beta chain, or a CD3 signaling subunit of the CD3 signaling complex.

In some embodiments, the second set of output molecules encoded by the replication-deficient herpesvirus comprises a T cell receptor (TCR) alpha chain or portion thereof; and/or a T cell receptor (TCR) beta chain or portion thereof. In some embodiments, the TCR alpha chain comprises a TCR alpha variable (TRAV) domain, wherein the TCR beta chain comprises a TCR beta variable (TRBV) domain, wherein covalent or non-covalent bonding of the TCR alpha and beta chains forms an TCR comprising an antigen-binding domain, wherein the antigen-binding domain is capable of binding to an antigen presentation complex, wherein the antigen presentation complex comprises an antigen and an antigen presentation protein. In some embodiments, the antigen presentation protein is a class I major histocompatibility complex (MHC-I) protein. In some embodiments, the antigen presentation protein is a class II major histocompatibility complex (MHC-II) protein. In some embodiments, the antigen is a neoantigen and/or a tumor-associated antigen. Neoantigen, as used herein, refers to an antigen that is encoded by cells harboring a mutation that changes the coding sequence of one or more genes. Mutations that change the coding sequence of a gene, such as a codon of the open reading frame or a splice site that leads to the inclusion of an intron in an open reading frame, lead to the expression of proteins not encoded by other cells of the body. A neoantigen may be a protein encoded by a mutated gene, or another molecule, such as a lipid or carbohydrate, that is modified by a mutated protein to have a structure that is not present in healthy cells. The expression of a neoantigen thus indicates that a cell harbors a mutation and is either a cancer cell, or may become cancerous. A tumor-associated antigen is an antigen that is found more often, or specifically, in tumors than in other sites of the body. A tumor-associated antigen may be an endogenous antigen that is more prevalent in tumor cells due to changes in gene expression or regulation, or a neoantigen.

In some embodiments, the antigen recognized by the TCR is a synthetic antigen, and the first set of output molecules encoded by the replication-competent herpesvirus comprises the synthetic antigen. Replication of the replication-competent herpesvirus in the tumor releases the synthetic antigen into the tumor microenvironment, which promotes activation of immune cells reprogrammed by the replication-deficient herpesvirus to recognize the synthetic antigen.

In some embodiments of the herpesvirus populations provided herein, the replication-deficient herpesvirus encodes a chimeric antigen receptor (CAR) as an output molecule. Expression of a CAR by an immune cell infected with a replication-deficient herpesvirus allows the herpesvirus to control the specificity of the infected immune cell, such that the infected immune cell is activated when the encoded CAR recognizes its target antigen. For example, the CAR encoded by the replication-deficient herpesvirus may be specific to a neoantigen or tumor-associated antigen, and thus the immune cell expressing the CAR will be activated by cells of the tumor and. If the cell is a cytotoxic cell, such as a CD8+ T cell, NK cell, or NK-T cell, the cell will kill cells expressing the target antigen, such as cancer cells.

A “chimeric antigen receptor” or “CAR” as used herein refers to a fused protein comprising an extracellular domain capable of binding to an antigen or ligand, a transmembrane domain typically derived from a polypeptide different from a polypeptide from which the extracellular domain is derived, and at least one intracellular domain. The molecular architecture of a CAR can be separated into three components: an extracellular ligand-recognizing domain (typically, although not exclusively, an scFv), a spacer and transmembrane domain (borrowed from other proteins such as antibody hinge regions and CD28 respectively), and intracellular immune signaling motifs (almost always the intracellular domain (ICD) of the TCR signaling component CD3ζ combined with one or more T cell costimulatory domains, such CD28 or 4-1BB).

As used herein, a “domain” is used interchangeably with the term “module” to mean one region in a polypeptide which is independent of other regions in the polypeptide, either functionally or structurally. The modules are described by name and or sequence. Exemplary sequences are provided herein. However, the claimed modules and structures are not limited to the exemplified sequences. The skilled artisan is familiar with extracellular domains, intracellular domains and transmembrane domains. Any such domains may be used in the constructs including naturally occurring versions of those domains, modified versions and synthetic versions. For instance the term CD3z refers to the zeta chain of CD3 and may include naturally occurring CD3z sequences as well as modified CD3z and synthetic CD3z sequences. Many sequences are included in publicly available databases.

The “extracellular domain” means any oligopeptide or polypeptide that can bind to a certain antigen or ligand. It may be a receptor, typically, although not exclusively, a single chain variable fragment (scFv). As used herein, a “single chain variable fragment” or “scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for preparing an scFv are known to a person skilled in the art.

In some embodiments, the antigen or ligand is a tumor antigen or ligand associated with the surface of a tumor cell. The antigen or ligand may be, for instance, any one or more of CD19, CD20, BCMA, CD22, CD38, CD138, mesothelin, VEGFR-2, CD4, CD5, CD30, CD22, CD24, CD25, CD28, CD30, CD33, CD47, CD52, CD56, CD80, CD81, CD86, CD123, CD171, CD276, B7H4, CD133, EGFR, GPC3; PMSA, CD3, CEACAM6, c-Met, EGFRvIII, ErbB2/HER-2, ErbB3/HER3, ErbB4/HER-4, EphA2,10a, IGF1R, GD2, 0-acetyl GD2, 0-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, NGFR, MCAM, TNFR1, TNFR2, PD1, PD-L1, PD-L2, HVEM, MAGE-A, NY-ESO-1, PSMA, RANK, ROR1, ROR-2, TNFRSF4, CD40, CD137, TWEAK-R, LTPR, LIFRP, LRP5, MUC1, TCRa, TCRp, TLR7, TLR9, PTCH1, WT-1, Robol, a, Frizzled, OX40, CD79b, and Notch-1-4. The extracellular domain of the CAR interacts with and specifically binds to the tumor antigen or ligand.

A “transmembrane domain” or “spacer” is a region which links the extracellular and intracellular domains and spans part or all of the membrane. It may be borrowed from other proteins such as antibody hinge regions and CD28 respectively. For instance, the transmembrane domain may be derived from a natural protein, or may be synthetic. The transmembrane domain derived from a natural protein can be obtained from any membrane-binding or transmembrane protein. For example, a transmembrane domain of a TCR-alpha or -beta chain, a CD3-zeta chain (CD3z), CD28, CD3-epsilon (CD3e), CD45, CD4, CD5, CD8, CD9, CD16, CD22, CD33, CD37, CD64, CD80, CD86, CD134, CD137, ICOS, CD154, or a GITR can be used. A synthetic transmembrane domain may comprise hydrophobic residues such as leucine and valine. In some embodiments, a triplet of phenylalanine, tryptophan and valine may be found at each end of the synthetic transmembrane domain.

The “intracellular domain” (ICD) means any oligopeptide or polypeptide which may function as a domain that transmits a signal to cause activation or inhibition of a biological process in a cell. These domains are intracellular immune signaling motifs, for example in typical CARs may be CD3z combined with one or more T cell costimulatory domains, such CD28 or 4-1BB. Exemplary ICD domains useful herein include but are not limited to: CD3E, CD3Z, CD3G, CD3D, CD79A, CD79B, DAP12, FCER1G, CD28, DAP10, CD137, CD134, ICOS, CD2, CD27, DNAM1, TIM1, CD30, DR3, HVEM, CRTAM, 2B4, CD84, CD19, CD40, CTLA4, PD1, TIM3, LAG3, BTLA, TIGIT, LILRB1, LILRB2, KIR3DL1, KIR3DL2, KIR2DL1, KIR2DL2, KIR2DL3, KIR2DL4, KIR2DL5, KIR3DL1, KIR3DL2, KIR3DL3, SIRPA, FCRL1, FCRL2, FCRL3, FCRL4, FCRL5, FCRL6, CD4, CD8A, CD8B, LAT, FCGR1A, FCGR2A, FCGR2B, FCGR3A, TLR1, TLR2, TLR3, TLR4, TLR5, TLR6, TLR7, TLR8, TLR9, TLR10, PILRB, NCR1, NCR2, NCR3, LY9, NKG2A, NKG2C, NKG2D, SLAMF6, SLAMF7, CD22, GITR, Human herpesvirus 8 type P K1 (K1_HEV8P), Epstein-Barr virus (strain B95-8) LMP2 (LMP2 EBVB9), Bovine leukemia virus (ENV_BLV), Mouse mammary tumor virus (strain C3H) (ENV_MMTVC), Rhesus monkey rhadinovirus H26-95 R1 (R1_RRV), African horse sickness virus (VP7_AHSV), IL-2RG, IL-2RB, IL-7R, IL-9R, IL-21R, IL-2, IL-7, IL-9, and IL-21.

Each of the domains disclosed herein may be combined with any other of the domains disclosed herein in any combination or order. The combinations may be of 2, 3, 4, 5, 6, 7, 8, 9, or 10 or more domains.

In some embodiments, the second set of output molecules encoded by the replication-deficient herpesvirus comprises a chimeric antigen receptor (CAR) that is capable of binding to an antigen. In some embodiments, the CAR comprises an extracellular single-chain variable fragment (scFv) of an antibody. In some embodiments, the CAR further comprises a hinge domain, a transmembrane domain, and one or more intracellular signal transduction domains. In some embodiments, one or more intracellular signal transduction domains are domains of a protein selected from the group consisting of CD28, CD3, and 4-1BB. In some embodiments, the antigen recognized by the CAR is a neoantigen and/or a tumor-associated antigen. In some embodiments, the antigen is a synthetic antigen, and the first set of output molecules encoded by the replication-competent herpesvirus comprises the synthetic antigen. Replication of the replication-competent herpesvirus in the tumor releases the synthetic antigen into the tumor microenvironment, which promotes activation of immune cells reprogrammed by the replication-deficient herpesvirus to recognize the synthetic antigen.

In some embodiments, the output molecule is a therapeutic molecule. A “therapeutic molecule” is a molecule that has therapeutic effects on a disease or condition, and may be used to treat a diseases or condition. Therapeutic molecules of the present disclosure may be nucleic acid-based or protein or polypeptide-based.

In some embodiments, nucleic acid-based therapeutic molecule may be an RNA interference (RNAi) molecule (e.g., a microRNA, siRNA, or shRNA) or a nucleic acid enzyme (e.g., a ribozyme). RNAi molecules and there use in silencing gene expression are familiar to those skilled in the art. In some embodiments, the RNAi molecule targets an oncogene. An oncogene is a gene that in certain circumstances can transform a cell into a tumor cell. An oncogene may be a gene encoding a growth factor or mitogen (e.g., c-Sis), a receptor tyrosine kinase (e.g., EGFR, PDGFR, VEGFR, or HER2/neu), a cytoplasmic tyrosine kinase (e.g., Src family kinases, Syk-ZAP-70 family kinases, or BTK family kinases), a cytoplasmic serine/threonine kinase or their regulatory subunits (e.g., Raf kinase or cyclin-dependent kinase), a regulatory GTPase (e.g., Ras), or a transcription factor (e.g., Myc). In some embodiments, the oligonucleotide targets Lipocalin (Lcn2) (e.g., a Lcn2 siRNA). One skilled in the art is familiar with genes that may be targeted for the treatment of cancer.

Non-limiting examples of protein or polypeptide-based therapeutic molecules include enzymes, regulatory proteins (e.g., immuno-regulatory proteins), antigens, antibodies or antibody fragments, and structural proteins. In some embodiments, the protein or polypeptide-based therapeutic molecules are for cancer therapy.

Suitable enzymes (for operably linking to a synthetic promoter) for some embodiments of this disclosure include, for example, oxidoreductases, transferases, polymerases, hydrolases, lyases, synthases, isomerases, and ligases, digestive enzymes (e.g., proteases, lipases, carbohydrases, and nucleases). In some embodiments, the enzyme is selected from the group consisting of lactase, beta-galactosidase, a pancreatic enzyme, an oil-degrading enzyme, mucinase, cellulase, isomaltase, alginase, digestive lipases (e.g., lingual lipase, pancreatic lipase, phospholipase), amylases, cellulases, lysozyme, proteases (e.g., pepsin, trypsin, chymotrypsin, carboxypeptidase, elastase), esterases (e.g. sterol esterase), disaccharidases (e.g., sucrase, lactase, beta-galactosidase, maltase, isomaltase), DNases, and RNases.

Non-limiting examples of antibodies and fragments thereof include: bevacizumab (AVASTIN®), trastuzumab (HERCEPTIN®), alemtuzumab (CAMPATH®, indicated for B cell chronic lymphocytic leukemia), gemtuzumab (MYLOTARG®, hP67.6, anti-CD33, indicated for leukemia such as acute myeloid leukemia), rituximab (RITUXAN®), tositumomab (BEXXAR®, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX®, indicated for ovarian cancer), edrecolomab (PANOREX®), daclizumab (ZENAPAX®), palivizumab (SYNAGIS®, indicated for respiratory conditions such as RSV infection), ibritumomab tiuxetan (ZEVALIN®, indicated for Non-Hodgkin's lymphoma), cetuximab (ERBITUX®), MDX-447, MDX-22, MDX-220 (anti-TAG-72), IOR-05, IOR-T6 (anti-CD1), IOR EGF/R3, celogovab (ONCOSCINT® OV103), epratuzumab (LYMPHOCIDE®), pemtumomab (THERAGYN®), Gliomab-H (indicated for brain cancer, melanoma). In some embodiments, the antibody is an antibody that inhibits an immune check point protein, e.g., an anti-PD-1 antibody such as pembrolizumab (Keytruda®) or nivolumab (Opdivo®), or an anti-CTLA-4 antibody such as ipilimumab (Yervoy®). Other antibodies and antibody fragments may be operably linked to a synthetic promoter, as provided herein.

A regulatory protein may be, in some embodiments, a transcription factor or a immunoregulatory protein. Non-limiting, exemplary transcriptional factors include: those of the NFkB family, such as Rel-A, c-Rel, Rel-B, p50 and p52; those of the AP-1 family, such as Fos, FosB, Fra-1, Fra-2, Jun, JunB and JunD; ATF; CREB; STAT-1, -2, -3, -4, -5 and -6; NFAT-1, -2 and -4; MAF; Thyroid Factor; IRF; Oct-1 and -2; NF-Y; Egr-1; and USF-43, EGR1, Sp1, and E2F1. Other transcription factors may be operably linked to a synthetic promoter, as provided herein.

As used herein, an immunoregulatory protein is a protein that regulates an immune response. Non-limiting examples of immunoregulatory include: antigens, adjuvants (e.g., flagellin, muramyl dipeptide), cytokines including interleukins (e.g., IL-2, IL-7, IL-15 or superagonist/mutant forms of these cytokines), IL-12, IFN-gamma, IFN-alpha, GM-CSF, FLT3-ligand), and immunostimulatory antibodies (e.g., anti-CTLA-4, anti-CD28, anti-CD3, or single chain/antibody fragments of these molecules). Other immunoregulatory proteins may be operably linked to a synthetic promoter, as provided herein.

As used herein, an antigen is a molecule or part of a molecule that is bound by the antigen-binding site of an antibody. In some embodiments, an antigen is a molecule or moiety that, when administered to or expression in the cells of a subject, activates or increases the production of antibodies that specifically bind the antigen. Antigens of pathogens are well known to those of skill in the art and include, but are not limited to parts (coats, capsules, cell walls, flagella, fimbriae, and toxins) of bacteria, viruses, and other microorganisms. Examples of antigens that may be used in accordance with the disclosure include, without limitation, cancer antigens, self-antigens, microbial antigens, allergens, and environmental antigens. Other antigens may be operably linked to a synthetic promoter, as provided herein.

In some embodiments, the antigen of the present disclosure is a cancer antigen. A cancer antigen is an antigen that is expressed preferentially by cancer cells (i.e., it is expressed at higher levels in cancer cells than on non-cancer cells) and, in some instances, it is expressed solely by cancer cells. Cancer antigens may be expressed within a cancer cell or on the surface of the cancer cell. Cancer antigens that may be used in accordance with the disclosure include, without limitation, MART-1/Melan-A, gp100, adenosine deaminase-binding protein (ADAbp), FAP, cyclophilin b, colorectal associated antigen (CRC)-C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4 and MAGE-05. The cancer antigen may be selected from the group consisting of GAGE-1, GAGE-2, GAGE-3, GAGE-4, GAGE-5, GAGE-6, GAGE-7, GAGE-8 and GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-3, SSX-4, SSX-5, SCP-1 and CT-7, CD20 and c-erbB-2. Other cancer antigens may be operably linked to a synthetic promoter, as provided herein.

In some embodiments, a protein or polypeptide-based therapeutic molecule is a fusion protein. A fusion protein is a protein comprising two heterologous proteins, protein domains, or protein fragments, that are covalently bound to each other, either directly or indirectly (e.g., via a linker), via a peptide bond. In some embodiments, a fusion protein is encoded by a nucleic acid comprising the coding region of a protein in frame with a coding region of an additional protein, without intervening stop codon, thus resulting in the translation of a single protein in which the proteins are fused together.

### Pharmaceutical Compositions and Methods of Use

In some aspects, the present disclosure provides a composition comprising one or more of the herpesviruses, or composition comprising a plurality of herpesviruses, provided herein. In some embodiments, the composition further comprises a pharmaceutically acceptable excipient. “Acceptable” means that the excipient (carrier) must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used for in vivo administration must be sterile. This is readily accomplished by, for example, filtration through sterile filtration membranes. The pharmaceutical compositions described herein may be placed into a container having a sterile access port, for example, an intravenous solution bag or vial having a stopper pierceable by a hypodermic injection needle.

In other embodiments, the pharmaceutical compositions described herein can be formulated for intramuscular injection, intravenous injection, intratumoral injection, or subcutaneous injection.

The pharmaceutical compositions described herein to be used in the present methods can comprise pharmaceutically acceptable carriers, buffer agents, excipients, salts, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some examples, the pharmaceutical composition described herein comprises lipid nanoparticles which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In other examples, the pharmaceutical composition described herein can be formulated in sustained-release format. Suitable examples of sustained-release preparations include semipermeable matrices of solid hydrophobic polymers containing the herpesvirus, the vector comprising the same, or the cell comprising the same, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

Suitable surface-active agents include, in particular, non-ionic agents, such as polyoxyethylenesorbitans (e.g., TWEEN™ 20, 40, 60, 80 or 85) and other sorbitans (e.g., SPAN™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable emulsions may be prepared using commercially available fat emulsions, such as INTRALIPID™, LIPOSYN™, INFONUTROLTM, LIPOFUNDINTM and LIPIPHYSAN™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets having a suitable size and can have a pH in the range of 5.5 to 8.0.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

Also provided herein are nucleic acid(s) and vector(s) comprising genomes of the herpesviruses described herein. Each component of the herpesvirus genome may be included in one or more (e.g., 2, 3 or more) nucleic acid molecules (e.g., vectors) and introduced into a cell. A “nucleic acid” is at least two nucleotides covalently linked together, and in some instances, may contain phosphodiester bonds (e.g., a phosphodiester “backbone”). A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine. Nucleic acids of the present disclosure may be produced using standard molecular biology methods (see, e.g., Green and Sambrook, Molecular Cloning, A Laboratory Manual, 2012, Cold Spring Harbor Press).

In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

In some aspects, the present disclosure provides methods comprising delivering any of the herpesviruses, herpesvirus genomes, or compositions comprising the herpesviruses or herpesvirus genomes to a cell, and optionally detecting an output molecule. In some embodiments, the herpesvirus is delivered to a cell by one or more vectors. A “vector” refers to a nucleic acid (e.g., DNA) used as a vehicle to artificially carry genetic material (e.g., an engineered nucleic acid) into a cell where, for example, it can be replicated and/or expressed. In some embodiments, a vector is an episomal vector (see, e.g., Van Craenenbroeck K. et al. Eur. J. Biochem. 267, 5665, 2000). Non-limiting examples of vectors include plasmids, bacterial artificial chromosomes (BACs), RNA replicons, viral vectors (e.g., rAAV, lentivirus). Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert.

The nucleic acids or vectors containing the herpesvirus genome may be delivered to a cell by any methods known in the art for delivering nucleic acids. For example, for delivering nucleic acids to a prokaryotic cell, the methods include, without limitation, transformation, transduction, conjugation, and electroporation. For delivering nucleic acids to a eukaryotic cell, methods include, without limitation, transfection, electroporation, and using viral vectors. In some embodiments, the replication-competent herpesvirus or herpesvirus genome and the replication-deficient herpesvirus or herpesvirus genome are delivered to the cells or subject by different nucleic acids or vectors. In some embodiments, there are different copy numbers of the herpesvirus genomes. In some embodiments, the ratio between the herpesvirus genomes is proportional. Proportional delivery of the herpesviruses or herpesvirus genomes means they are delivered at a ratio. In some embodiments, the ration between the nucleic acids or vectors carrying the replication-competent herpesvirus genome and the nucleic acids or vectors carrying the replication-deficient herpesvirus genome is 1:1, 1:2, 1:3, 1:4, 1:5, 1:6, 1:7, 1:8, 1:9, 1:10, 2:3, 2:5, 2:7, 2:9, 3:4, 3:5, 3:7, 3:8, 3:10, 4:5, 4:7, 4:9, 4:10, 5:6, 5:7, 5:8, 5:9, 6:7, 7:8, 7:9, 7:10, 8:9, 9:10, 10:1, 9:1, 8:1, 7:1, 6:1, 5:1, 4:1, 3:1, 2:1, 3:2, 5:2, 7:2, 9:2, 4:3, 5:3, 7:3, 8:3, 10:3, 5:4, 7:4, 9:4, 10:4, 6:5, 7:5, 8:5, 9:5, 7:6, 8:7, 9:7, 10:7, 9:8, or 10:9.

Detecting an output molecule, as used herein, refers to measuring the amount or presence of the output molecule present in or produced by a herpesvirus or herpesvirus genome comprising a nucleic acid sequence encoding the output molecule. Methods of measuring the amount or presence of an output molecule are well known in the art, with non-limiting methods of measurement including ELISA, PCR, qRT-PCR, fluorescence-activated cell sorting (FACS), microscopy, and fluorescent microscopy.

Also provided herein are cells comprising the herpesvirus or the vectors encoding the same as described herein. A “cell” is the basic structural and functional unit of all known independently living organisms. It is the smallest unit of life that is classified as a living thing. Some organisms, such as most bacteria, are unicellular (consist of a single cell). Other organisms, such as humans, are multicellular.

In some embodiments, a cell for use in accordance with the present disclosure is a eukaryotic cell, which comprises membrane-bound compartments in which specific metabolic activities take place, such as a nucleus. Examples of eukaryotic cells for use in accordance with the invention include, without limitation, mammalian cells, insect cells, yeast cells (e.g., Saccharomyces cerevisiae) and plant cells. In some embodiments, the eukaryotic cells are from a vertebrate animal. In some embodiments, the cell is a mammalian cell. In some embodiments, the cell is a human cell. In some embodiments, the cell is from a rodent, such as a mouse or a rat. Examples of vertebrate cells for use in accordance with the present disclosure include, without limitation, reproductive cells including sperm, ova and embryonic cells, and non-reproductive cells, immune, kidney, lung, spleen, lymphoid, cardiac, gastric, intestinal, pancreatic, muscle, bone, neural, brain and epithelial cells. Stem cells, including embryonic stem cells or induced pluripotent stem cells, can also be used.

In some embodiments, the cell is a diseased cell. A “diseased cell,” as used herein, refers to a cell whose biological functionality is abnormal, compared to a non-diseased (normal) cell. In some embodiments, the diseased cell is a cancer cell.

In some embodiments, the cell comprising a genome of a replication-deficient herpesvirus is a T cell, T cell precursor, NK cell, or NK cell precursor. In some embodiments, the cell is a CD4+ T cell. In some embodiments, the cell is a CD8+ T cell. In some embodiments, the cell is an NK-T cell. In some embodiments, the genome of the replication-deficient herpesvirus is integrated into a chromosome of the cell. The nucleic acid sequence of an integrated viral genome in a chromosome is contiguous with the nucleic acid sequence of the rest of the chromosome. Replication of chromosomes containing integrated viral genomes, and subsequent cell division, results in both daughter cells containing chromosomes with integrated viral genomes. Thus, the replication-deficient herpesvirus is maintained during mitosis despite a lack of active viral replication.

In some embodiments, the cell is a cell used for recombinant protein production. Non-limiting examples of recombinant protein producing cells are Chinese hamster ovary (CHO) cells, human embryonic kidney (HEK)-293 cells, verda reno (VERO) cells, nonsecreting null (NS0) cells, human embryonic retinal (PER.C6) cells, Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells.

In some aspects, the present disclosure provides methods of treating a disease or disorder, the method comprising delivering any of the herpesviruses or herpesvirus genomes, or compositions comprising herpesviruses or herpesvirus genomes, provided herein to a subject in need thereof, wherein the output molecule is a therapeutic molecule that treats the disease or disorder.

In some embodiments of the methods provided herein, the subject is a human. In some embodiments, the subject has or is at risk of developing cancer. In some embodiments, the cancer is selected from the group consisting of melanoma, carcinoma, breast cancer, lung cancer, kidney cancer, bladder cancer, pancreatic cancer, gastric cancer, stomach cancer, liver cancer, colorectal cancer, ovarian cancer, uterine cancer, esophageal cancer, brain cancer, leukemia, and lymphoma.

### EXAMPLES

### Example 1: Design and Validation of Engineered HSV-1 for Expression of Genetic Programs in Cancer Cells and Immune Cells

The goal of the research described in this Example was to directly lyse tumor cells and prime adaptive immune system using engineered HSV-1 carrying anti-tumor genetic programs. Each of two distinct anti-tumor programs were encoded in the genome of replicating HSV-1 and non-replicating HSV-1. The program encoded in replicating HSV-1 is activated only when tumor cells are infected. The program drives selective replication in tumor cells resulting in lysis of tumor cells and release of tumor specific antigens. During this process, it expresses cytokines and antibodies to modulate tumor microenvironment to better accommodate priming of adaptive immune systems. The second program is encoded in the genome of non-replicating HSV-1 and it directly infects immune cells including CD4+ or CD8+ cells, expressing TCR, CAR, cytokines, and/or antibodies to prime the adaptive immune system.

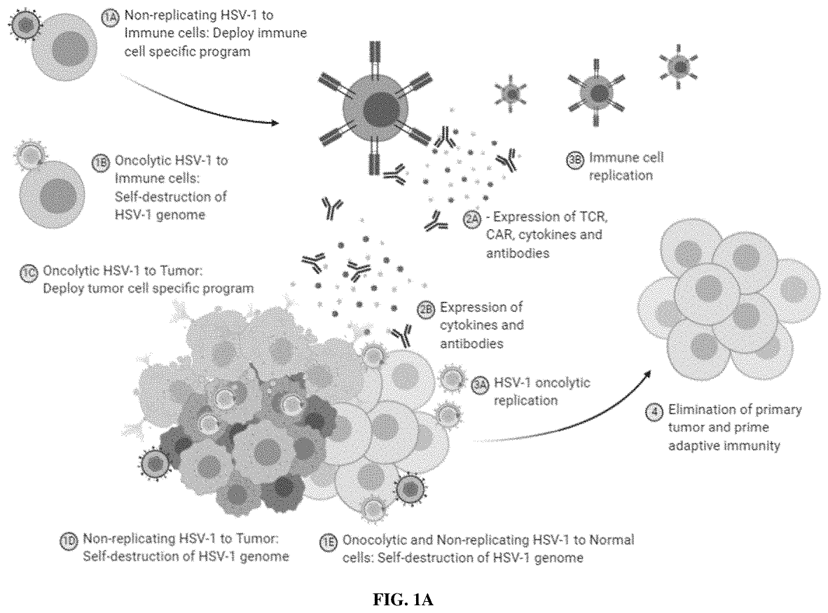

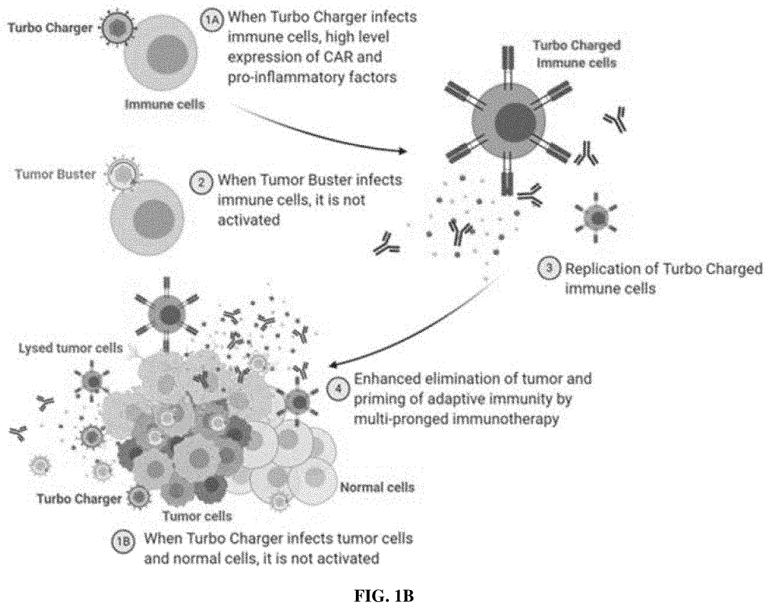

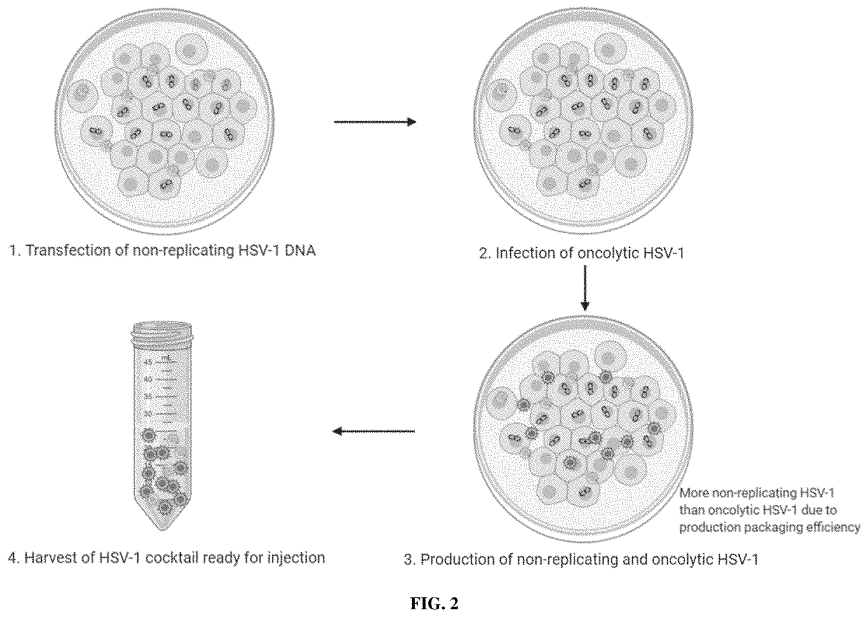

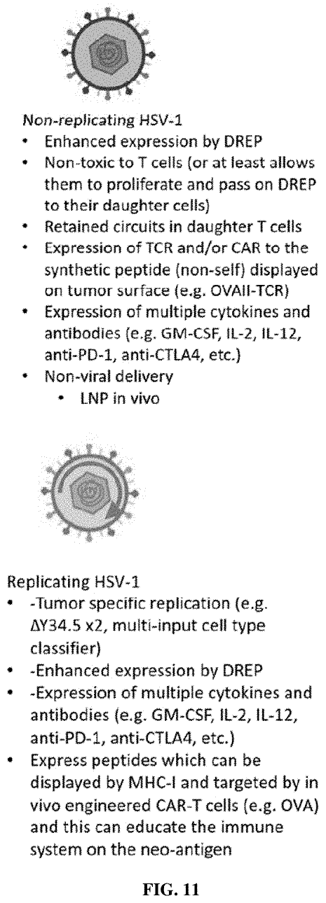

Herpesvirus populations containing two engineered HSV-1 viruses were designed for the simultaneous delivery of genetic programs for cancer therapy. A first virus, a non-replicating HSV-1, was designed to deliver an immune cell-specific program to immune cells, such as T cells, to express a TCR, CAR, cytokines, and/or antibodies. A second virus, a replicating HSV-1, was designed as an oncolytic virus to replicate in tumors, killing cancer cells while also expressing cytokines and/or antibodies to counteract the immunosuppressive environment of the tumor (FIGS. 1A-1B, FIG. 11). Herpesvirus populations were prepared by transfecting the DNA genome of the non-replicating HSV-1 into cells, infecting the cells with replicating HSV-1, which also facilitates replication of non-replicating HSV-1 genomes from co-infected cells, allowing replication to occur, and harvesting an HSV-1 cocktail that is suitable for injection to a subject (FIG. 2).

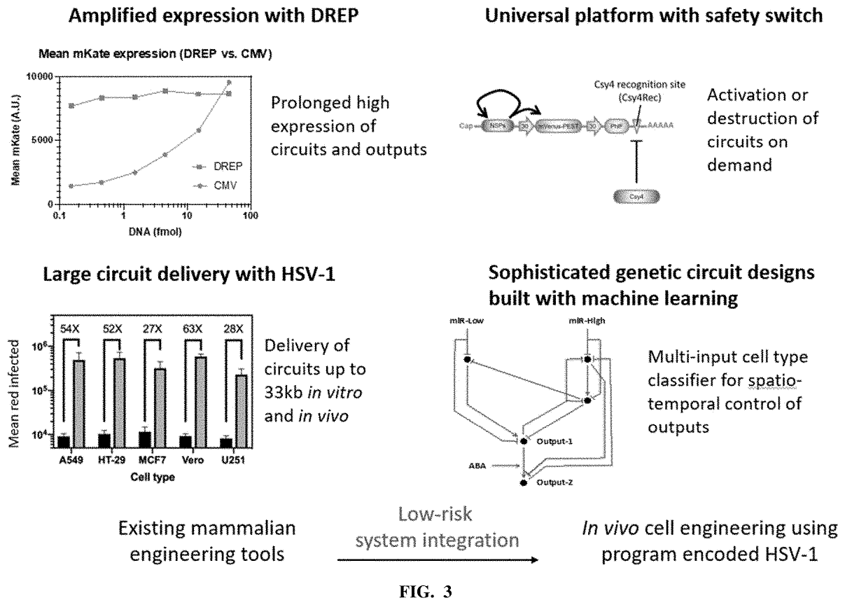

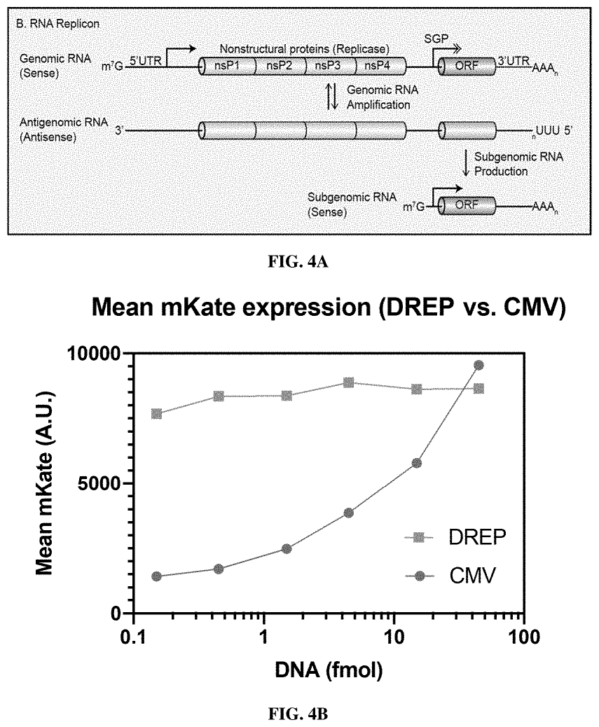

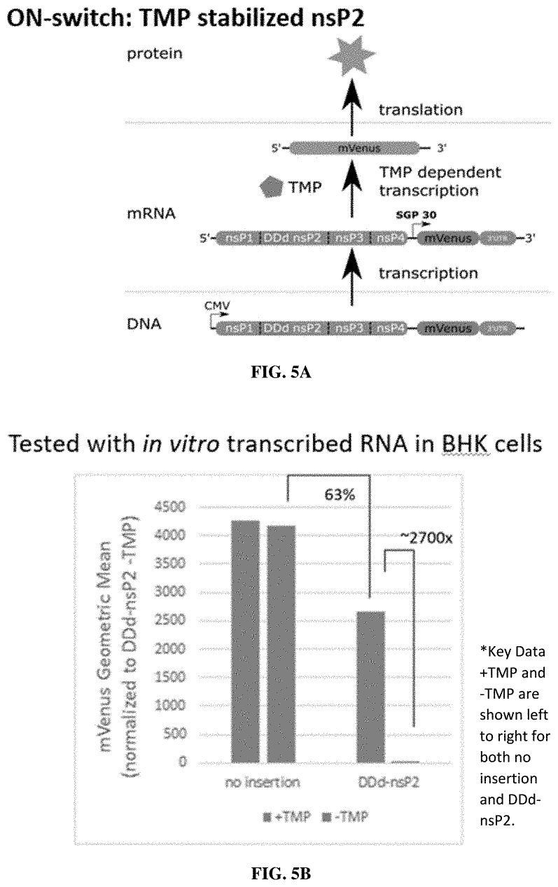

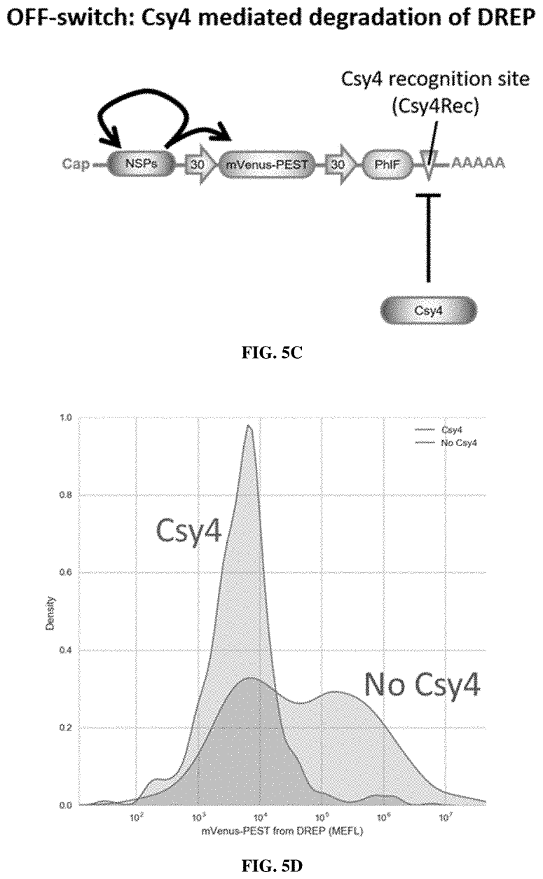

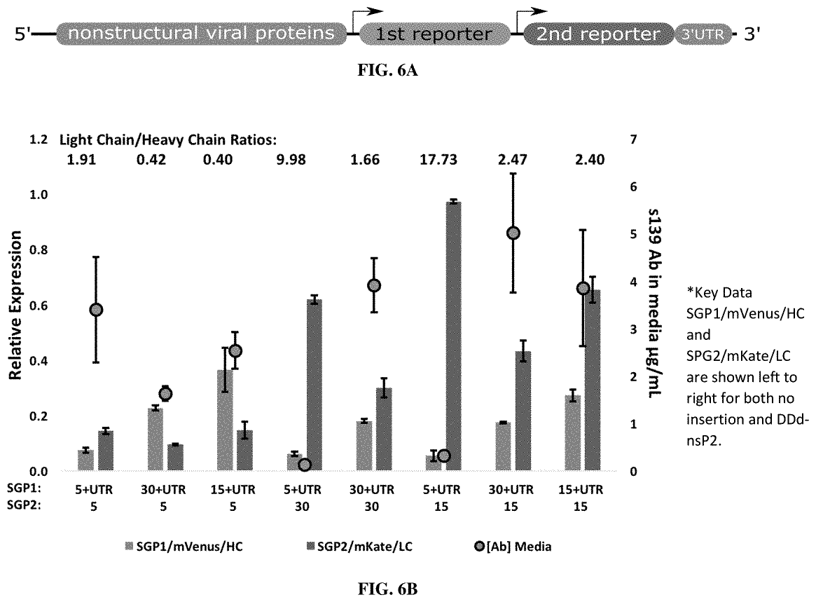

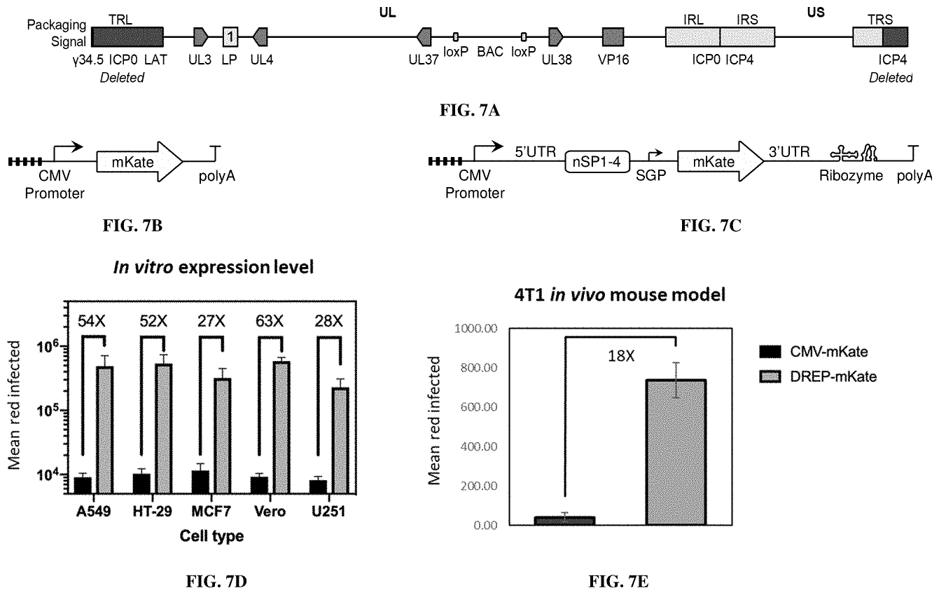

Engineered herpesvirus populations have several advantages that make them useful for the delivery of genetic programs (FIG. 3). First, encoding a DNA-launched RNA replicon (DREP) in the herpesvirus genome allows for robust amplification of the RNA containing the genetic program in the cell (FIGS. 4A-4B). Second, the genetic program is engineered to contain one or more safety switches for control of gene expression. For example, a destabilization domain in one of the proteins required to replicate the RNA replicon serves as an ON switch, so that replication occurs only in the presence of a stabilizing molecule, such as TMP or OHT-1 (FIGS. 5A-5B). Additionally, an endoribonuclease recognition site on the RNA replicon serves as an OFF switch, so that the RNA replicon can be degraded in the presence of the corresponding endoribonuclease, such as Csy4 (FIGS. 5C-5D). Third, the large size of herpesvirus genomes, allows for the delivery of large genetic programs, up to 33 kb in the case of HSV-1 (FIGS. 7A-7E). Finally, the large size of herpesvirus genomes allows for the inclusion of sophisticated genetic circuits with multiple inputs, such as a given miRNA profile characterized by the presence of one or more miRNAs and the absence of others, to restrict the expression of output molecules to a desired context. Additionally, expression of each molecule can be differentially controlled through the use of different subgenomic promoters or the addition of additional components to the genetic programs (FIGS. 6A-6B).

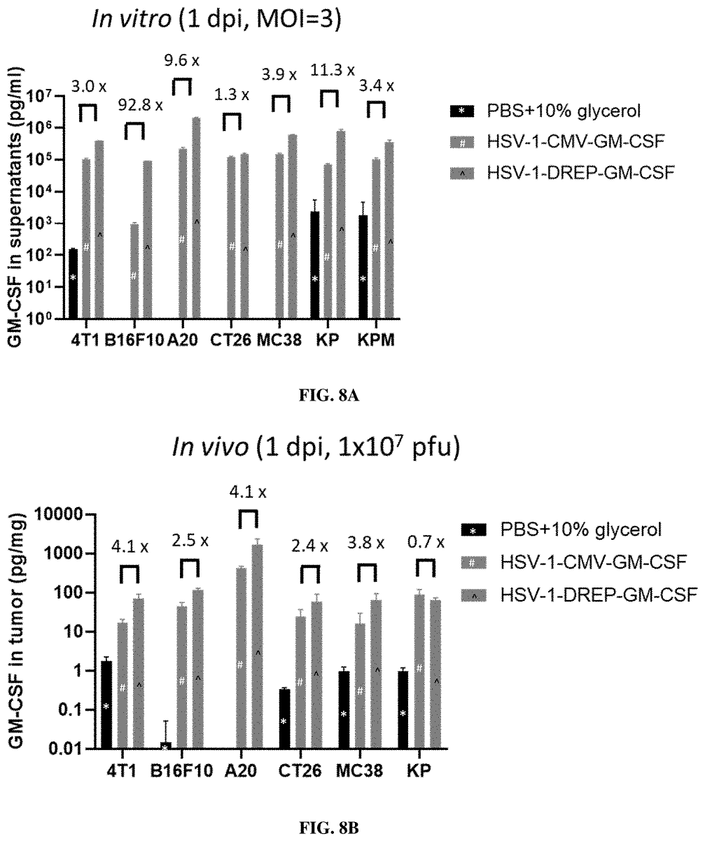

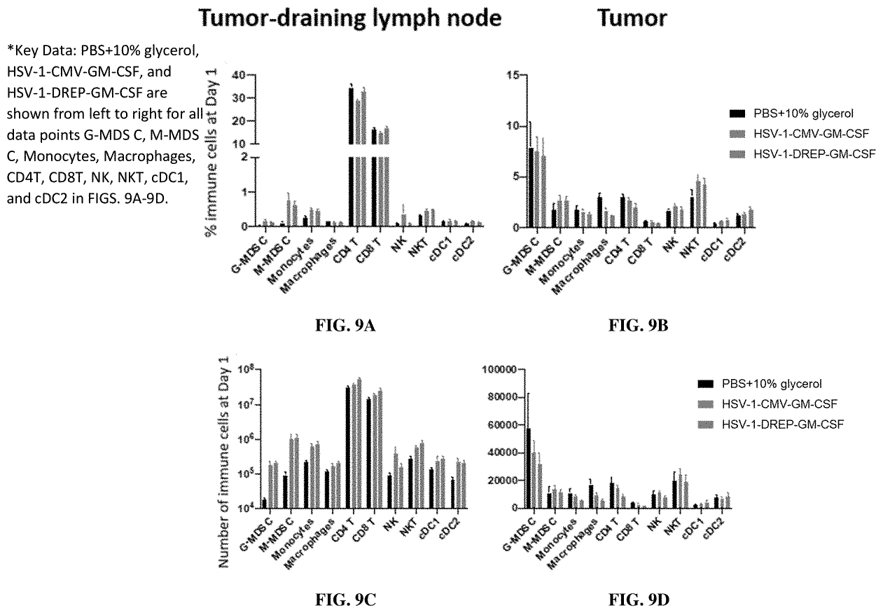

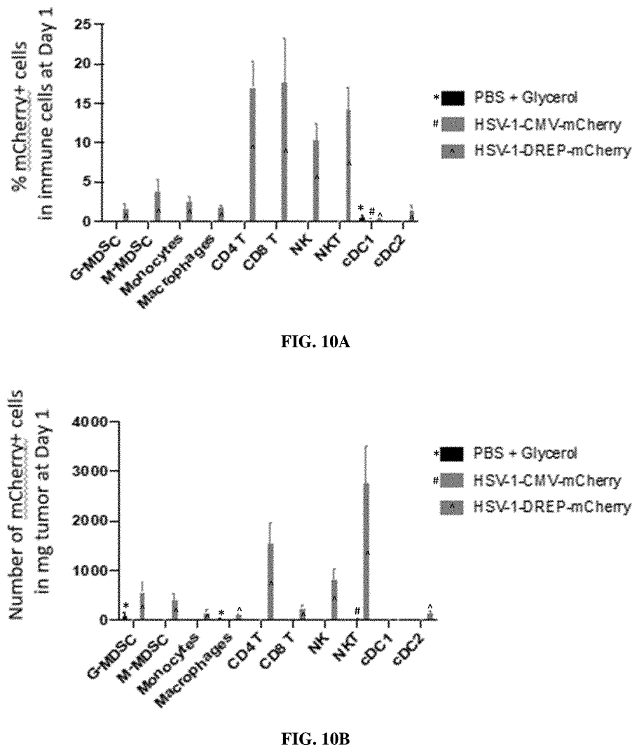

The ability of engineered herpesviruses to deliver genetic programs and express encoded output molecules was tested both in vitro and in vivo. Engineered HSV-1 viruses with a genome encoding GM-CSF under the control of a CMV promoter, or containing a DREP encoding GM-CSF, were used to inoculate multiple cancer cell lines. One day post-inoculation, the amount of GM-CSF in cell culture supernatants was quantified for each inoculated group of cells. In each cancer cell line tested, HSV-1 containing the GM-CSF-encoding DREP consistently induced more GM-CSF production than HSV-1 encoding GM-CSF under control of CMV promoter in the genome (FIG. 8A). The same viruses were administered to mice containing engrafted cancer cells of the same types tested in cell culture, and the concentrations of GM-CSF in tumors were quantified one day post-inoculation. With one exception, HSV-1 containing the GM-CSF-encoding DREP led to higher concentrations of GM-CSF in tumors than HSV-1 encoding GM-CSF in the genome under the control of CMV promoter (FIG. 8B). Tumors and tumor-draining lymph nodes in these mice were also analyzed by flow cytometry to quantify the abundance, in terms of proportions and absolute numbers, of multiple types of immune cells in each lymph node and tumor (FIGS. 9A-9D). Finally, to determine the ability of engineered herpesviruses to express genetic programs in immune cells, HSV-1 viruses containing DREPs encoding mCherry, or encoding mCherry in the genome under the control of a CMV promoter, were administered to mice. One day post-inoculation, cells were obtained from the mice and analyzed by flow cytometry to quantify the proportion of mCherry+ immune cells of each type (FIG. 10A). Additionally, the absolute number of mCherry+ immune cells of each type per milligram of tumor mass was quantified by flow cytometry (FIG. 10B).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms. All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A herpesvirus population comprising:
(i) a replication-competent herpesvirus comprising a cancer cell state classifier, wherein the cancer cell state classifier comprises:
(a) a cancer cell sensor circuit comprising a first constitutive promoter operably linked to a nucleic acid encoding:
(1) one or more target sequences for a first set of cancer input miRNAs; and
(2) a nucleic acid sequence encoding one or more of a first set of repressors; and
(b) a cancer cell signal circuit comprising a first subgenomic promoter operably linked to a nucleic acid encoding:
(1) one or more of a first set of output molecules; and
(2) a first repressor recognition sequence that is capable of being bound by the first repressor, wherein the first repressor is capable of binding to the first repressor recognition sequence to prevent expression of the first set of output molecules;
(ii) a replication-deficient herpesvirus comprising an immune cell state classifier, wherein the immune cell state classifier comprises:
(a) an immune cell sensor circuit comprising a second constitutive promoter operably linked to a nucleic acid encoding:
(1) one or more target sequences for a first set of immune cell input miRNAs; and
(2) a nucleic acid sequence encoding one or more of a second set of repressors; and
(b) an immune cell signal circuit comprising a second subgenomic promoter operably linked to a nucleic acid encoding:
(1) one or more of a second set of output molecules; and
(2) a second repressor recognition sequence that is capable of being bound by the second repressor, wherein the second repressor is capable of binding to the second repressor recognition sequence to prevent expression of the second set of output molecules.

2. The herpesvirus population of claim 1, wherein:
(i) the first set of repressors comprises one or more of a first set of repressor RNAi molecules, and the first repressor recognition sequence comprises one or more target sequences for one or more of the first set of repressor RNAi molecules, optionally wherein the first set of repressor RNAi molecules comprises a first set of repressor miRNAs and the first repressor recognition sequence comprises one or more target sequences for one or more of the first set of repressor miRNAs; and/or
(ii) the second set of repressors comprises one or more of a second set of repressor RNAi molecules, and the second repressor recognition sequence comprises one or more target sequences for one or more of the second set of repressor RNAi molecules, optionally wherein the second set of repressor RNAi molecules comprises a second set of repressor miRNAs and the second repressor recognition sequence comprises one or more target sequences for one or more of the second set of repressor miRNAs,
optionally wherein the first set of repressor miRNAs does not comprise any miRNAs of the second set of repressor miRNAs,
optionally wherein the second set of repressor miRNAs does not comprise any miRNAs of the first set of repressor miRNAs.

3-4. (canceled)

5. The herpesvirus population of claim 1, wherein:
(i) the first repressor recognition sequence comprises a first endoribonuclease recognition sequence, and the first set of repressors comprises a first endoribonuclease that is capable of cleaving the first endoribonuclease recognition sequence; and/or
(ii) the second repressor recognition sequence comprises a second endoribonuclease recognition sequence, and the second set of repressors comprises a second endoribonuclease that is capable of cleaving the second endoribonuclease recognition sequence,
optionally the first endoribonuclease is not capable of cleaving the second endoribonuclease ribonuclease recognition sequence,
optionally wherein the second endoribonuclease is not capable of cleaving the first endoribonuclease recognition sequence,
optionally wherein the first endoribonuclease is a first CRISPR endoribonuclease selected from the group consisting of Cas6, Csy4, CasE, Cse3, LwaCas13a, PspCas13b, RanCas13b, PguCas13b, and RfxCas13d,
optionally wherein the second endoribonuclease is a second CRISPR endoribonuclease selected from the group consisting of Cas6, Csy4, CasE, Cse3, LwaCas13a, PspCas13b, RanCas13b, PguCas13b, and RfxCas13d,
optionally wherein the first endoribonuclease and the second endoribonuclease are different endoribonucleases.

6-8. (canceled)

9. The herpesvirus population of claim 1, wherein:
(i) the cancer cell signal circuit further comprises one or more target sequences for a second set of cancer input miRNAs; and/or
(ii) the immune cell signal circuit further comprises one or more target sequences for a second set of immune cell input miRNAs.

10. The herpesvirus population of claim 1, wherein the cancer cell state classifier comprises up to 5 kb, up to 10 kb, up to 15 kb, up to 20 kb, up to 25 kb, up to 30 kb, up to 31 kb, up to 32 kb, up to 33 kb, up to 34 kb, up to 35 kb, up to 36 kb, up to 37 kb, up to 38 kb, up to 39 kb, or up to 50 kb,
wherein the immune cell state classifier comprises up to 5 kb, up to 10 kb, up to 15 kb, up to 20 kb, up to 25 kb, up to 30 kb, up to 31 kb, up to 32 kb, up to 33 kb, up to 34 kb, up to 35 kb, up to 36 kb, up to 37 kb, up to 38 kb, up to 39 kb, or up to 50 kb.

11-13. (canceled)

14. The herpesvirus population of claim 1, wherein:
(i) the first constitutive promoter is an hEF1a promoter; and/or
(ii) the second constitutive promoter is an hEF1a promoter.

15. The herpesvirus population of claim 1, wherein the cancer cell state classifier is a DNA encoding a cancer cell RNA replicon, wherein the cancer cell RNA replicon comprises the cancer cell sensor circuit and the cancer cell signal circuit,
wherein the immune cell state classifier is a DNA encoding an immune cell RNA replicon, wherein the immune cell RNA replicon comprises the immune cell sensor circuit and the immune cell signal circuit,
optionally wherein the cancer cell RNA replicon comprises a nucleic acid sequence encoding one or more proteins that are capable of replicating the cancer cell RNA replicon,
optionally wherein the immune cell RNA replicon comprises a nucleic acid sequence encoding one or more proteins that are capable of replicating the immune cell RNA replicon,
optionally wherein one or more of the proteins that are capable of replicating the cancer cell RNA replicon and/or the immune cell RNA replicon comprise a destabilization domain,
optionally wherein the destabilization domain is selected from the group consisting of PEST, a destabilization domain from E. coli dihydrofolate reductase, a destabilization domain derived from human FK506-binding protein (FKBP), and a destabilization domain derived from FKBP-rapamycin-binding (FRB) protein.

16-19. (canceled)

20. The herpesvirus population of claim 1, wherein the replication-competent herpesvirus is a herpesvirus selected from the group consisting of herpes simplex virus (HSV)-1, HSV-2, Varicella-Zoster virus (VZV), Epstein-Barr virus (EBV), human cytomegalovirus (CMV), roseolovirus, and Kaposi's sarcoma herpesvirus (KSHV),
wherein the replication-competent herpesvirus is a herpesvirus selected from the group consisting of herpes simplex virus (HSV)-1, HSV-2, Varicella-Zoster virus (VZV), Epstein-Barr virus (EBV), human cytomegalovirus (CMV), roseolovirus, and Kaposi's sarcoma herpesvirus (KSHV),
optionally wherein the replication-competent herpesvirus is HSV-1 and/or the replication-deficient herpesvirus is HSV-1,
optionally wherein the replication-competent herpesvirus and the replication-deficient herpesvirus are HSV-1.

21-23. (canceled)

24. The herpesvirus population of claim 1, wherein:
(i) the first set of output molecules comprises one or more cytokines; and/or
(ii) the second set of output molecules comprises one or more cytokines,
optionally wherein the first set of output molecules comprises one or more cytokines selected from the group consisting of IL-1β, IL-2, IL-4, IL-6, IL-7, IL-10, IL-12, IL-15, IL-17, IL-18, IFN-γ, TNF-α, and GM-CSF,
optionally wherein the second set of output molecules comprises one or more cytokines selected from the group consisting of IL-1β, IL-2, IL-4, IL-6, IL-7, IL-10, IL-12, IL-15, IL-17, IL-18, IFN-γ, TNF-α, and GM-CSF,
optionally wherein the first set of output molecules comprises IL-2, IL-12, and GM-CSF and the second set of output molecules comprises IL-2, IL-12, and GM-CSF.

25-27. (canceled)

28. The herpesvirus population of claim 1, wherein:
(i) the first set of output molecules comprises one or more antibodies or antigen-binding fragments thereof; and/or
(ii) the second set of output molecules comprises one or more antibodies or antigen-binding fragments thereof,
optionally wherein one or more antibodies are selected from the group consisting of an anti-PD-1 antibody, an anti-PD-L1 antibody, and an anti-CTLA-4 antibody, or antigen-binding fragments thereof,
optionally wherein one or more of the antibodies or antigen-binding fragments thereof is a monoclonal antibody, a chimeric antibody, a humanized antibody, a human engineered antibody, a human antibody, a single chain antibody (scFv), or an antibody fragment.

29-31. (canceled)

32. The herpesvirus population of claim 1, wherein the second set of output molecules comprises:
(i) a T cell receptor (TCR) alpha chain or portion thereof; and/or
(ii) a T cell receptor (TCR) beta chain or portion thereof,
wherein covalent or non-covalent bonding of the TCR alpha and beta chains forms an TCR comprising an antigen-binding domain, wherein the antigen-binding domain is capable of binding to an antigen presentation complex, wherein the antigen presentation complex comprises an antigen and an antigen presentation protein,
optionally wherein the TCR alpha chain comprises a TCR alpha variable (TRAV) domain,
optionally wherein the TCR beta chain comprises a TCR beta variable (TRBV) domain,
optionally wherein the antigen is a neoantigen and/or tumor-associated antigen,
optionally wherein the antigen presentation protein is a class I major histocompatibility complex (MHC-I) protein or a class II major histocompatibility complex (MHC-I) protein.

33-35. (canceled)

36. The herpesvirus population of claim 1, wherein the second set of output molecules comprises a chimeric antigen receptor (CAR) or portion thereof, wherein the CAR or portion thereof is capable of binding to an antigen,
optionally wherein the antigen is a neoantigen and/or tumor-associated antigen,
optionally wherein the CAR comprises an extracellular single-chain variable fragment (scFv) of an antibody,
optionally wherein the CAR further comprises a hinge domain, a transmembrane domain, and one or more intracellular signal transduction domains,
optionally wherein one or more intracellular signal transduction domains are domains of a protein selected from the group consisting of CD28, CD3, and 4-1BB.

37-40. (canceled)

41. The herpesvirus population of claim 1, wherein the first set of output molecules comprises a neoantigen and/or a tumor-associated antigen.

42. The herpesvirus population of claim 1, wherein the first set of output molecules comprises the synthetic antigen.

43. The herpesvirus population of claim 1, wherein:
(i) the first set of output molecules comprises one or more immunostimulatory ligands; and/or
(ii) the second set of output molecules comprises one or more immunostimulatory ligands,
optionally wherein one or more immunostimulatory ligands are selected from the group consisting of a LysM-containing protein, flagellin-grp170, cowpea mosaic virus (CPMV) small coat protein, and CPMV large coat protein,
optionally wherein the LysM-containing protein comprises an amino acid sequence with at least 90%, at least 95%, or up to 100% sequence identity to the amino acid sequence of SEQ ID NO: 14,
optionally wherein the flagellin-grp170 comprises an amino acid sequence with at least 90%, at least 95%, or up to 100% sequence identity to the amino acid sequence of SEQ ID NO: 19,
optionally wherein the CPMV small coat protein comprises an amino acid sequence with at least 90%, at least 95%, or up to 100% sequence identity to the amino acid sequence of SEQ ID NO: 22,
optionally wherein the CPMV large coat protein comprises an amino acid sequence with at least 90%, at least 95%, or up to 100% sequence identity to the amino acid sequence of SEQ ID NO: 23.

44-50. (canceled)

51. A nucleic acid encoding a genome of the replication-competent herpesvirus or the replication-deficient herpesvirus of claim 1.

52. (canceled)

53. A vector comprising the nucleic acid of claim 51, optionally wherein the vector is formulated in a lipid nanoparticle.

54-55. (canceled)

56. A cell comprising a genome of the replication-deficient herpesvirus of claim 1,
optionally wherein the cell is a T cell, T cell precursor, NK cell, or NK cell precursor,
optionally wherein the cell is a CD4+ T cell,
optionally wherein the cell is a CD8+ T cell,
optionally wherein the genome of the replication-deficient herpesvirus is integrated into a chromosome of the cell.

57-60. (canceled)

61. A pharmaceutical composition comprising a pharmaceutically acceptable excipient and
the herpesvirus population of claim 1.

62. A method comprising administering to a subject the herpesvirus population of claim 1,
optionally wherein the subject is a human,
optionally wherein the subject has or is at risk of developing cancer,
optionally wherein the cancer is selected from the group consisting of melanoma, squamous cell cancer, small-cell lung cancer, non-small cell lung cancer, lung cell adenocarcinoma, squamous lung cell carcinoma, peritoneal cancer, hepatocellular cancer, gastrointestinal cancer, esophageal cancer, stomach cancer, pancreatic cancer, glioblastoma, cervical cancer, ovarian cancer, liver cancer, bladder cancer, hepatoma, breast cancer, colon cancer, colorectal cancer, endometrial carcinoma, uterine carcinoma, salivary gland carcinoma, kidney cancer, prostate cancer, vulval cancer, thyroid cancer, hepatic carcinoma, gastric cancer, head-and-neck cancer, leukemia, and lymphoma.

63-65. (canceled)
